(19) United States
(12) Patent Application Publication
(10) Pub. No.: US 2021/0038324 A1
(43) Pub. Date: Feb. 11, 2021

(54) GUIDED SURGERY APPARATUS AND METHOD

(71) Applicant: Trophy, Croissy-Beaubourg (FR)

(72) Inventors: Jean-Marc Inglese, Bussy-Saint-Georges (FR); Eamonn Boyle, Croissy-Beaubourg (FR); Arnaud Capri, Villiers-sur-Marne (FR); Yannick Glinec, Montevrain (FR)

(21) Appl. No.: 17/078,645

(22) Filed: Oct. 23, 2020

Related U.S. Application Data
(63) Continuation of application No. 16/078,971, filed on Aug. 22, 2018, now abandoned.
Application No. 16/078,971 is a continuation of application No. PCT/IB2016/000325, filed on Feb. 26, 2016.

(51) Int. Cl. A61B 34/20; A61B 5/00; G06T 17/00; A61B 90/00; G06T 7/70; A61B 6/14; A61C 1/08; A61C 3/02; A61C 9/00; G06T 19/00

(52) U.S. Cl. CPC A61B 34/20; A61B 5/0088; A61B 5/0062; G06T 17/00; A61B 90/37; G06T 7/70; A61B 2034/105; A61C 1/082; A61C 3/02; A61C 9/0046; G06T 19/006; G06T 2210/41; A61B 6/14

(57) ABSTRACT

Method and apparatus embodiments can acquire and update a 3-D surface of a dentition in real time by replacing the corresponding portion of the 3-D surface of the dentition with the contents of newly acquired 3-D image. In certain embodiments, the position of the 3-D scanning device relative to the 3-D surface of the dentition can be determined in real time by comparing the size and the shape of the overlap to the cross-section of the field-of-view of the 3-D scanning device, where the size and the shape of the overlap of the newly acquired 3-D image is used to determine the distance and the angles from which the 3-D image was acquired relative to the 3-D surface of the dentition.

### TECHNICAL FIELD

The disclosure relates generally to 3-D diagnostic imaging and more particularly to apparatus and methods for guided surgery with dynamic updating of image display according to treatment progress.

### BACKGROUND

Guided surgery techniques have grown in acceptance among medical and dental practitioners, allowing more effective use of image acquisition and processing utilities and providing image data that is particularly useful to the practitioner at various stages in the treatment process. Using guided surgery tools, for example, the practitioner can quickly check the positioning and orientation of surgical instruments and verify correct angles for incision, drilling, and other invasive procedures where accuracy can be a particular concern.

The capability for radiographic volume imaging, using tools such as cone-beam computed tomography (CBCT), has been particularly helpful for improving the surgical planning process. Intraoral volume imaging, for example, makes it possible for the practitioner to study bone and tissue structures of a patient in detail, such as for implant positioning. Surgical planning tools, applied to the CBCT volume image, help the practitioner to visualize and plan where drilling needs to be performed and to evaluate factors such as amount of available bone structure, recommended drill depth, clearance obstructions, and other variables. Symbols for drill paths or other useful markings can be superimposed onto the volume image display so that these can be viewed from different perspectives and used for guidance during the procedure.

One problem with radiographic volume imaging for surgical guidance relates to update. Once a drilling or other procedure has begun, and as it continues, the volume image that was originally used for surgical planning can become progressively less accurate as a guide to ongoing work. Removal or displacement of tissue may not be accurately represented in the volume image display, so that further guidance may not be as reliable as the initial surgical plan.

A number of conventional surgical guidance imaging systems address the update problem by providing fiducial markers of some type, positioned on the patient's skin or attached to adjacent teeth or nearby structures, or positioned on the surgical instrument itself. Fiducial markers are then used as guides for updating the volume image content. There are drawbacks with this type of approach, however, including obstruction or poor visibility, added time and materials needed for mounting the fiducial markers or marking the surface of the patient, patient discomfort, and other difficulties. Moreover, fiducial markers only provide reference landmarks for the patient anatomy or surgical instrumentation; additional computation is still required in order to update the volume display to show procedure progress. The display itself becomes increasingly less accurate as to actual conditions. Similar limitations relate to inaccurate surface depiction; when using the radiographic image content, changes to the surface contour due to surgical procedures, such as due to incision, drilling, tooth removal, or implant placement, are not displayed.

Among solutions proposed for surgical guidance, fiducial markers, and related techniques for combined image content are those described in U.S. Patent Application Publication No. 2006/0281991 by Fitzpatrick, et al.; U.S. Patent Application Publication No. 2008/0183071 by Strommer et al.; U.S. Patent Application Publication No. 2008/0262345 by Fichtinger et al.; U.S. Patent Application Publication No. 2012/0259204 by Carrat et al.; U.S. Patent Application Publication No. 2010/0168562 by Zhao et al.; U.S. Patent Application Publication No. 2006/0165310 by Newton; U.S. Patent Application Publication No. 2013/0063558 by Phipps; U.S. Patent Application Publication No. 2011/0087332 by Bojarski et al.; U.S. Pat. No. 6,122,541 to Cosman et al.; U.S. Patent Application Publication No. 20100298712 by Pelissier et al.; Patent application WO 2012/149548 A2 by Siewerdsen et al.; Patent application WO 2012/068679 by Dekel et al.; Patent application WO 2013/144208 by Daon; and Patent application WO 2010/086374 by Lavalee et al.

Structured light imaging is one familiar technique that has been successfully applied for surface characterization. In structured light imaging, a pattern of illumination is projected toward the surface of an object from a given angle. The pattern can use parallel lines of light or more complex periodic features, such as sinusoidal lines, dots, or repeated symbols, and the like. The light pattern can be generated in a number of ways, such as using a mask, an arrangement of slits, interterometric methods, or a spatial light modulator, such as a Digital Light Processor from Texas Instruments Inc., Dallas, Tex. or similar digital micromirror device. Multiple patterns of light may be used to provide a type of encoding that helps to increase robustness of pattern detection, particularly in the presence of noise. Light reflected or scattered from the surface is then viewed from another angle as a contour image, taking advantage of triangulation in order to analyze surface information based on the appearance of contour lines or other patterned illumination.

Intraoral structured light imaging is now becoming a valuable tool for the dental practitioner, who can obtain this information by scanning the patient's teeth using an inexpensive, compact intraoral scanner, such as the Model CS3500 Intraoral Scanner from Carestream Dental, Atlanta, Ga. However, structured light imaging only provides information about the surface contour at the time of scanning. This information can quickly become inaccurate as a dental procedure progresses.

There is a need for providing automated surgical guidance apparatus and methods that can help practitioners to plan and execute procedures such as the placement of implants and other devices. Capable imaging tools for both internal structures and contour imaging have been developed. However, there is a need to make this information accessible to the practitioner during the surgery procedure, without requiring cumbersome display apparatus and without distracting the practitioner from concentration on the surgical treatment site.

### SUMMARY

It is an object of the present disclosure to advance the art of dental surgical guidance. Apparatus and methods can be provided that take advantage of volume image reconstruction and contour surface image characterization to present real-time guidance images to the dental surgical practitioner.

Another aspect of this application is to address, in whole or in part, at least the foregoing and other deficiencies in the related art.

It is another aspect of this application to provide, in whole or in part, at least the advantages described herein.

These objects are given only by way of illustrative example, and such objects may be exemplary of one or more embodiments of the disclosure. Other desirable objectives and advantages inherently achieved by the may occur or become apparent to those skilled in the art. The invention is defined by the appended claims.

According to one aspect of the disclosure, there is provided a method for acquiring and updating a 3-D surface of a dentition that can include a) acquiring a collection of 3-D image content of the dentition from a different points of view using a 3-D scanning device; b) gradually forming the 3-D surface of the dentition using a matching algorithm that aggregates 3-D images from the 3-D image content based on a determination of overlap of each 3-D image relative to the 3-D surface of the dentition; wherein for each newly acquired 3-D image, i) when the newly acquired 3-D image partly overlaps with the 3-D surface of the dentition, augmenting the 3-D surface of the dentition with a portion of the newly acquired 3-D image that does not overlap with the 3-D surface of the dentition, and ii) when the newly acquired 3-D image completely overlaps with the 3-D surface of the dentition, updating the 3-D surface of the dentition in real time by replacing the corresponding portion of the 3-D surface of the dentition with the contents of newly acquired 3-D image, where the corresponding portion of the 3-D surface of the dentition no longer contributes to the updated 3D surface of the dentition. In one aspect, the position of the 3-D scanning device relative to the 3-D surface of the dentition can be determined in real time by comparing the size and the shape of the overlap to the cross-section of the field-of-view of the 3-D scanning device, where the size and the shape of the overlap of the newly acquired 3-D image is used to determine the distance and the angles from which the 3-D image was acquired relative to the 3-D surface of the dentition.

According to one aspect of the disclosure, there is provided a method for updating display of a dentition to a practitioner that can include obtaining 3-D surface contour image content that includes a dentition treatment region; obtaining radiographic volume image content that includes the dentition treatment region; combining the 3-D surface contour image content and the radiographic volume image content into a single 3-D virtual model that comprises the dentition treatment region; obtaining instructions that define a surgical treatment plan related to the treatment region; repeating the steps of a1) acquiring new 3-D contour images of the dentition treatment region that include physical dental objects in the dentition treatment region from different points of view using a 3-D scanning device, and a2) updating the 3-D surface of the dentition treatment region in real time by replacing the corresponding portion of the 3-D surface of the dentition treatment region with the contents of the newly acquired 3-D contour images, where the corresponding portion of the 3-D surface of the dentition no longer contributes to the updated 3D surface of the dentition; and repeating the steps of b1) sensing the position of a surgical instrument mounted to the 3-D scanning device at a surgical site within the dentition treatment region, relative to the single 3-D virtual model; b2) updating the single 3-D virtual model according to the surgical treatment plan; b3) determining a field of view of the practitioner and detecting a tooth surface in the dentition treatment region in the practitioner's field of view and displaying at least a portion of the updated single 3-D virtual model onto the field of view and oriented to the field of view and registered to the actual tooth surface as seen from the practitioners' field of view.

### BRIEF DESCRIPTION OF THE DRAWINGS

The foregoing and other objects, features, and advantages of the disclosure will be apparent from the following more particular description of the embodiments of the disclosure, as illustrated in the accompanying drawings.

The elements of the drawings are not necessarily to scale relative to each other.

FIG. 1 is a schematic block diagram of an imaging system for surgical guidance according to an embodiment of the present disclosure.

FIG. 2 is a schematic block diagram of a scanning apparatus.

FIG. 3 is a schematic diagram that shows how patterned light is used for obtaining surface contour information by a scanner.

FIG. 4 shows surface imaging of a tooth or other feature using a pattern with multiple lines of light.

FIG. 5 is a perspective view that shows a portion of a point cloud, with connected vertices forming a mesh.

FIG. 6A is a schematic view that shows overlaid structured light images obtained over a treatment region.

FIG. 6B is a schematic view that shows overlaid structured light images obtained over a region that is adjacent to and at least slightly overlaps the treatment region.

FIG. 6C shows extension of the 3-D mesh according to a newly acquired surface contour image.

FIG. 6D shows the extended 3-D mesh of FIG. 6C.

FIG. 6E shows how newly acquired mesh portion can be used to update an existing mesh.

FIG. 6F shows an updated mesh that incorporates newly scanned mesh content.

FIG. 7 is an example display view showing details of an exemplary surgical plan.

FIG. 8A shows a schematic view of a head-mounted device (HMD) as worn by a practitioner according to an embodiment of the present disclosure.

FIG. 8B shows a schematic view of a head-mounted device (HMD) as worn by a practitioner according to an embodiment of the present disclosure, with augmented reality display components shown.

FIG. 8C is a schematic diagram that shows how the head-mounted device can define a field of view for the dental practitioner.

FIG. 9 is a schematic diagram that shows components of an HMD for augmented reality viewing.

FIG. 10 is a schematic diagram that shows a surgical instrument that includes sensing circuitry that may include a camera or image sensing device, according to an embodiment of the present disclosure.

FIG. 11 is a schematic diagram that shows a surgical instrument coupled to a camera for contour imaging.

FIG. 12 is a logic flow diagram showing an exemplary workflow for surgical guidance using augmented reality imaging according to an embodiment of the present disclosure.

FIG. 13 is a logic flow diagram that shows steps for image combination.

FIG. 14 shows an exemplary display view for guidance in a dental procedure.

FIGS. 15A and 15B are schematic views that show imaging components associated with a surgical instrument.

FIG. 15C is a schematic view that shows an alternate embodiment for a surgical instrument having two sensing circuits to detect instrument position using triangulation.

FIG. 16 is a logic flow diagram that shows a sequence for providing real-time update to displayed image content according to the surgical procedure.

FIG. 17 is a logic flow diagram that shows a sequence for providing display content that supports a dental surgical procedure.

FIG. 18 shows a simplified schematic view of a depth-resolved imaging apparatus for intraoral imaging.

FIGS. 19 and 20 each show a swept-source OCT (SS-OCT) apparatus using a programmable filter according to an embodiment of the present disclosure.

FIG. 21 is a schematic diagram that shows data acquired during an OCT scan.

FIG. 22 shows an OCT B-scan for two teeth, with and without fluid content.

FIG. 23 is a logic flow diagram showing contour image rendering with compensation for fluid according to an embodiment of the present disclosure.

FIGS. 24A and 24B show image examples with segmentation of blood and saliva.

FIG. 25 is a logic flow diagram that shows a sequence that can be used for imaging a tooth surface according to an embodiment of the present disclosure.

### DESCRIPTION OF EXEMPLARY EMBODIMENTS

The following is a detailed description of exemplary embodiments, reference being made to the drawings in which the same reference numerals identify the same elements of structure in each of the several figures.

Where they are used, the terms “first”, “second”, and so on, do not necessarily denote any ordinal or priority relation, but may be used for more clearly distinguishing one element or time interval from another.

The term “exemplary” indicates that the description is used as an example, rather than implying that it is an ideal. The terms “subject” and “object” may be used interchangeably to identify the object of an optical apparatus or the subject of an image.

The term “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the terms “pixel” and “voxel” may be used interchangeably to describe an individual digital image data element, that is, a single value representing a measured image signal intensity. Conventionally an individual digital image data element is referred to as a voxel for 3-dimensional or volume images and a pixel for 2-dimensional (2-D) images. For the purposes of the description herein, the terms voxel and pixel can generally be considered equivalent, describing an image elemental datum that is capable of having a range of numerical values. Voxels and pixels have attributes of both spatial location and image data code value.

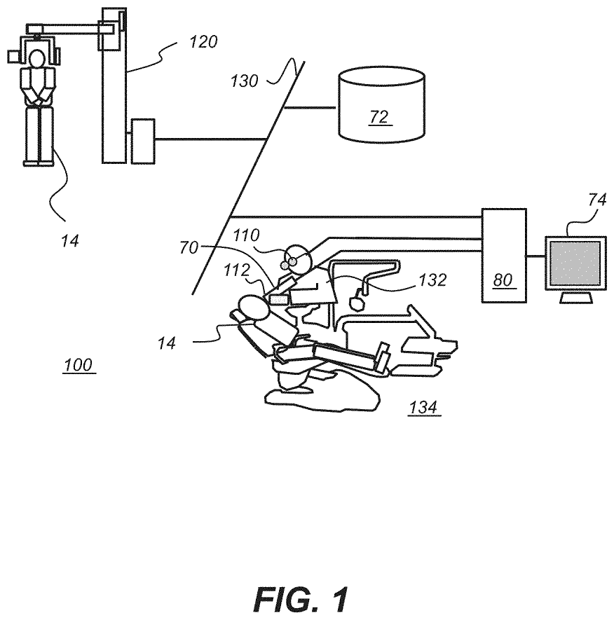

Volumetric imaging data is obtained from a volume radiographic imaging apparatus such as a computed tomography system, CBCT system 120 as shown in FIG. 1, or other imaging system that obtains volume image content related to bone and other internal tissue structure. The volume image content can be obtained by processing a sequence of 2-D projection images, each 2-D projection image acquired at a different angle with relation to the subject. Processing can use well known reconstruction algorithms such as back projection, FDK processing, or algebraic reconstruction methods, for example.

In the context of the present disclosure, a 3-D image or “3-D image content” can include:
(i) volume image content that includes information about the composition of material that lies within a three-dimensional object and includes material lying below the surface of an object. By volume image or “volume image content” is meant the acquired and processed image data that is needed in order to form voxels for 3-D image presentation. Volume image content can be obtained from a radiographic volumetric imaging apparatus such as a cone-beam computed tomography (CBCT) system, for example. Voxels that are used for a displayed slice or view of an object are defined from the stored volume image content according to image presentation characteristics defined by the viewer such as perspective angle, image slice, and other characteristics of the 3-D imaging environment.
(ii) surface contour image content that provides data for characterizing a surface, such as surface structure, curvature, and contour characteristics, but is not able to provide information on material that lies below the surface. Contour imaging data or surface contour image data can be obtained from a dental 3-D scanning device such as an intra-oral structured light imaging apparatus or from an imaging apparatus that obtains structure information related to a surface from a sequence of 2-D reflectance images obtained using visible light, near-infrared light, or ultraviolet light wavelengths. Alternate techniques for contour imaging such as dental contour imaging can include structured light imaging as well as other known techniques for characterizing surface structure, such as feature tracking by triangularization, structure from motion photogrammetry, time-of-flight imaging, and depth from focus imaging, for example. Contour image content can also be extracted from volume image content, such as by identifying and collecting only those voxels that represent surface tissue, for example.

“Patterned light” is used to indicate light that has a predetermined spatial pattern, such that the light has one or more features such as one or more discernable parallel lines, curves, a grid or checkerboard pattern, or other features having areas of light separated by areas without illumination. In the context of the present disclosure, the phrases “patterned light” and “structured light” are considered to be equivalent, both used to identify the light that is projected onto the head of the patient in order to derive contour image data.

In the context of the present disclosure, a single projected line of light is considered a “one dimensional” pattern, since the line has an almost negligible width, such as when projected from a line laser, and has a length that is its predominant dimension. Two or more of such lines projected side by side, either simultaneously or in a scanned arrangement, can be used to provide a two-dimensional pattern.

The terms “3-D model” and “point cloud” may be used synonymously in the context of the present disclosure. The dense point cloud is formed using techniques familiar to those skilled in the volume imaging arts for forming a point cloud and relates generally to methods that identify, from the point cloud, vertex points corresponding to surface features. The dense point cloud can be generated using the reconstructed contour data from one or more reflectance images. Dense point cloud information serves as the basis for a polygon model at high density, such as can be used for a 3-D surface for dentition including the teeth and gum surface.

In the context of the present disclosure, the terms “virtual view” and “virtual image” are used to connote computer-generated or computer-processed images that are displayed to the viewer. The virtual image that is generated can be formed by the optical system using a number of well-known techniques and this virtual image can be formed by the display optics using convergence or divergence of light. A magnifying glass, as a simple example, provides a virtual image of its object. A virtual image is not formed on a display surface but is formed by an optical system that provides light at angles that give the appearance of an actual object at a position in the viewer's field of view; the object is not actually at that position. With a virtual image, the apparent image size is independent of the size or location of a display surface. The source object or source imaged beam for a virtual image can be small. In contrast to systems that project a real image on a screen or display surface, a more realistic viewing experience can be provided by forming a virtual image that is not formed on a display surface but formed by the optical system; the virtual image appears to be some distance away and appears, to the viewer, to be superimposed onto or against real-world objects in the field of view (FOV) of the viewer.

In the context of the present disclosure, an image is considered to be “in register” with a subject that is in the field of view when the image and subject are visually aligned from the perspective of the observer. As the term “registered” is used in the current disclosure, a registered feature of a computer-generated or virtual image is sized, positioned, and oriented on the display so that its appearance represents the planned or intended size, position, and orientation for the corresponding object, correlated to the field of view of the observer. Registration is in three dimensions, so that, from the view perspective of the dental practitioner/observer, the registered feature is rendered at the position and angular orientation that is appropriate for the patient who is in the treatment chair and within the visual field of the observing practitioner. Thus, for example, where the computer-generated feature is a registered virtual image for a drill hole or drill axis for a patient's tooth, and where the observer is looking into the mouth of the patient, the display of the drill hole or axis can appear as if superimposed or overlaid within the mouth sized, oriented and positioned at the actual tooth for drilling and/or dentition surgical site as seen from the detected perspective of the observer. The relative opacity of superimposed content and/or registered virtual content can be modulated to allow ease of visibility of both the real-world view and the virtual image content that is superimposed thereon. In addition, because the virtual image content can be digitally generated, the superimposed content and/or registered content can be removed or its appearance changed in order to provide improved visibility of the real-world scene in the field of view or in order to provide various types of information to the practitioner.

In the context of the present disclosure, the term “real-time image” refers to an image that is actively acquired from the patient or displayed during a procedure in such a way that the image reflects the actual status of the procedure with no more than a few seconds' lag time, with imaging system response time as the primary factor in determining lag time. Thus, for example, a real-time display of drill position would closely approximate the actual drill position or targeted position, offset in time only by the delay time needed to process and display the image after being acquired or processed from stored image data.

In the context of the present disclosure, the term “highlighting” for a displayed feature has its conventional meaning as is understood to those skilled in the information and image display arts. In general, highlighting uses some form of localized display enhancement to attract the attention of the viewer. Highlighting a portion of an image, such as an individual tooth or a set of teeth or other structure(s) can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher sharpness or contrast.

In the context of the present disclosure, the terms “viewer”, “operator”, and “user” are considered to be equivalent and refer to the viewing practitioner, technician, or other person who views and manipulates a contour image that is formed from a combination of multiple structured light images on a display monitor.

A “viewer instruction”, “operator instruction”, or “operator command” can be obtained from explicit commands entered by the viewer or may be implicitly obtained or derived based on some other user action, such as making an equipment setting, for example. With respect to entries entered on an operator interface, such as an interface using a display monitor and keyboard, for example, the terms “command” and “instruction” may be used interchangeably to refer to an operator entry.

In the context of the present disclosure, the term “at least one of” is used to mean one or more of the listed items can be selected. The term “about” indicates that the value listed can be somewhat altered, as long as the alteration does not result in nonconformance of the process or structure to the illustrated embodiment.

In the context of the present disclosure, the term “coupled” is intended to indicate a mechanical association, connection, relation, or linking between two or more components, such that the disposition of one component affects the spatial disposition of a component to which it is coupled. For mechanical coupling, two components need not be in direct contact, but can be linked through one or more intermediary components.

Embodiments of the present disclosure are directed to the need for improved status tracking and guidance for the practitioner during surgical procedure using a volume image and augmented reality display, wherein the display of the volume image content is continuously refreshed to update the progress of the drill or other surgical instrument. Advantageously, radiographic volume image content for internal structures can be combined with surface contour image content for outer surface features, to form a virtual model or a single 3-D virtual model so that the combination forms the 3-D image content that displays to the practitioner as a virtual model that provides a surgical plan that can be continuously updated as work on the patient progresses. Certain exemplary embodiments can register the updtable single 3-D virtual model to the detected field of view of the practitioner.

The schematic block diagram of FIG. 1 shows an imaging system 100 that provides static and/or dynamic feedback to a surgical practitioner 132 at a surgical facility 134 to aid and facilitate a variety of procedures for a treatment region of a patient 14 including but not limited to: endodontics, oral surgery, periodontics, restorative dentistry, orthodontics, implantology, hygienic treatment, and maxillofacial surgery. Imaging system 100 is shown as a set of imaging apparatus connected on a network 130. Imaging system 100 includes a radiographic volume imaging apparatus, such as a cone beam computerized tomography (CBCT) system 120 that obtains radiographic volume image content by scanning patient 14. The radiographic volume image content is stored in a memory 72 that is accessible to other processors on network 130.

Real time feedback can be presented to the practitioner on the conventional display 74 monitor or on a wearable display such as a head-mounted device (HMD) 110. A scanning imaging apparatus 70 is disposed to continuously monitor the progress of a surgical instrument 112 as the treatment procedure progresses.

Alternately, 3-D image content can be obtained by acquiring and processing radiographic image data from a scanned cast, such as a molded appliance obtained from the patient.

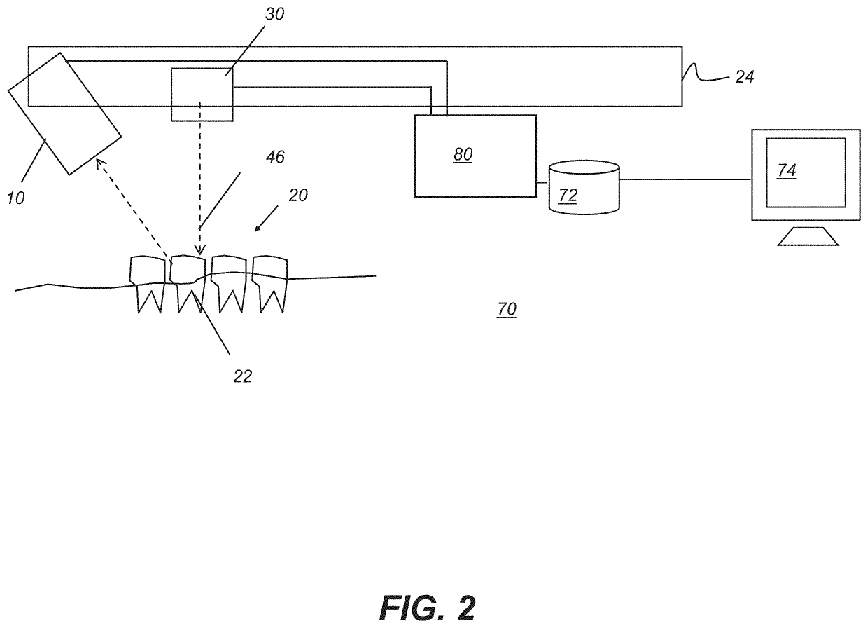

FIG. 2 is a schematic diagram showing an imaging apparatus 70, a scanner for scanning, projecting, and imaging to characterize surface contour using structured light patterns 46. Imaging apparatus 70 is an example of an intra-oral 3-D scanning device. Imaging apparatus 70 uses a handheld camera 24 for image acquisition according to an embodiment of the present disclosure. A control logic processor 80, or other type of computer that may be part of camera 24 controls the operation of an illumination array 10 that generates the structured light and controls operation of an imaging sensor array 30. Image data from surface 20, such as from a tooth 22, is obtained from imaging sensor array 30 and stored in memory 72. Control logic processor 80, in signal communication with camera 24 components of the scanner that acquire the image, processes the received image data from the scanner and stores the mapping in memory 72. The resulting image from memory 72 is then optionally rendered and displayed on a display 74. Memory 72 may also include a display buffer.

In structured light imaging, a pattern of lines, or other structured pattern, is projected from illumination array 10 toward the surface of an object from a given angle. The projected pattern from the surface is then viewed from another angle as a contour image, taking advantage of triangulation in order to analyze surface information based on the appearance of contour lines. Phase shifting, in which the projected pattern is incrementally shifted spatially for obtaining additional measurements at the new locations, is typically applied as part of structured light imaging, used in order to complete the contour mapping of the surface and to increase overall resolution in the contour image.

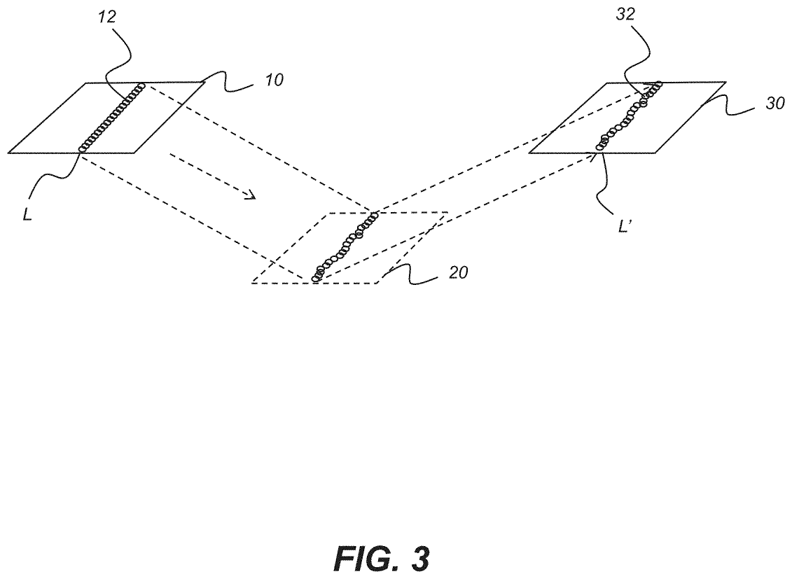

The schematic diagram of FIG. 3 shows, with the example of a single line of light L, how patterned light is used for obtaining surface contour information by a scanner using a handheld camera or other portable imaging device. A mapping is obtained as illumination array 10 directs a pattern of light onto a surface 20 and a corresponding image of a line L′ is formed on an imaging sensor array 30. Each pixel 32 on imaging sensor array 30 maps to a corresponding pixel 12 on illumination array 10 according to modulation by surface 20. Shifts in pixel position, as represented in FIG. 3, yield useful information about the contour of surface 20. It can be appreciated that the basic pattern shown in FIG. 3 can be implemented in a number of ways, using a variety of illumination sources and sequences and using one or more different types of sensor arrays 30. Illumination array 10 can utilize any of a number of types of arrays used for light modulation, such as a liquid crystal array or digital micromirror array, such as that provided using the Digital Light Processor or DLP device from Texas Instruments, Dallas, Tex. This type of spatial light modulator is used in the illumination path to change the light pattern as needed for the mapping sequence.

By projecting and capturing images that show structured light patterns that duplicate the arrangement shown in FIG. 3 multiple times, the image of the contour line on the camera simultaneously locates a number of surface points of the imaged object. This speeds the process of gathering many sample points, while the plane of light (and usually also the receiving camera) is laterally moved in order to “paint” some or all of the exterior surface of the object with the plane of light.

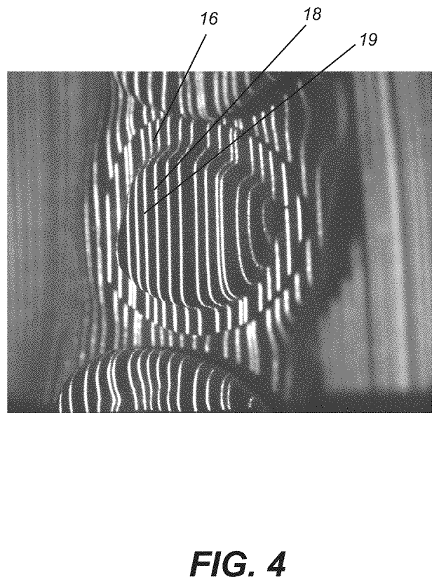

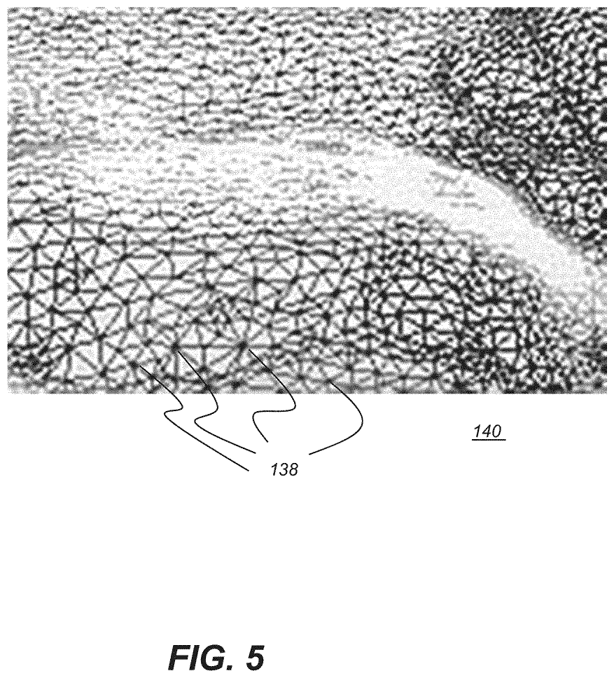

FIG. 4 shows surface imaging using a pattern with multiple lines of light. Incremental shifting of the line pattern and other techniques help to compensate for inaccuracies and confusion that can result from abrupt transitions along the surface, whereby it can be difficult to positively identify the segments that correspond to each projected line. In FIG. 4, for example, it can be difficult over portions of the surface to determine whether line segment 16 is from the same line of illumination as line segment 18 or adjacent line segment 19. By knowing the instantaneous position of the scanner and the instantaneous position of the line of light within a object-relative coordinate system when the image was acquired, a computer equipped with appropriate software can use triangulation methods to compute the coordinates of numerous illuminated surface points. As the plane is moved to intersect eventually with some or all of the surface of the object, the coordinates of an increasing number of points are accumulated. As a result of this image acquisition, a point cloud of vertex points or vertices can be identified and used to characterize the surface contour. FIG. 5 shows a portion of a point cloud, with connected vertices 138 to form a mesh 140. The points or vertices 138 in the point cloud then represent actual, measured points on the three dimensional surface of an object.

The surface data for surface contour characterization, also referred to as a surface data set, is obtained by a process that derives individual points from the structured images, typically in the form of a point cloud, wherein the individual points represent points along the surface of the imaged tooth or other feature. A close approximation of the surface object can be generated from a point cloud by connecting adjacent points and forming polygons, each of which closely approximates the contour of a small portion of the surface. Alternately, surface data can be obtained from the volumetric voxel data, such as data from a CBCT apparatus. Surface voxels can be identified and distinguished from voxels internal to the volume using threshold techniques or boundary detection using gray levels, for example. Thus, the term “surface” can be used to indicate data that is obtained either by processing volumetric data from a radiography-based system or as contour data acquired from a scanner or camera using structured or patterned light. While different file formats can be used to represent surface data, a number of systems that show surface features of various objects use the STL (STereoLithography) file format originally used with computer-aided design systems for 3D.

It should also be noted that image content for forming the mesh 140 of FIG. 5 can alternately be obtained from a scanner and associated imaging devices that use other methods for characterizing the surface contour, as described in more detail subsequently.

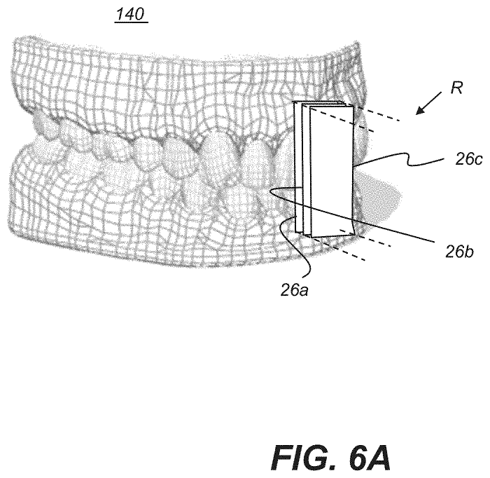

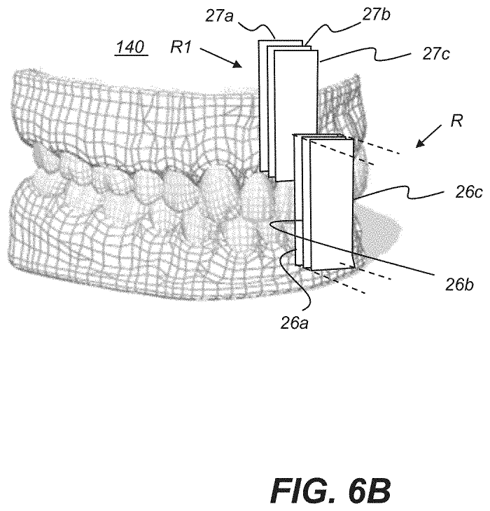

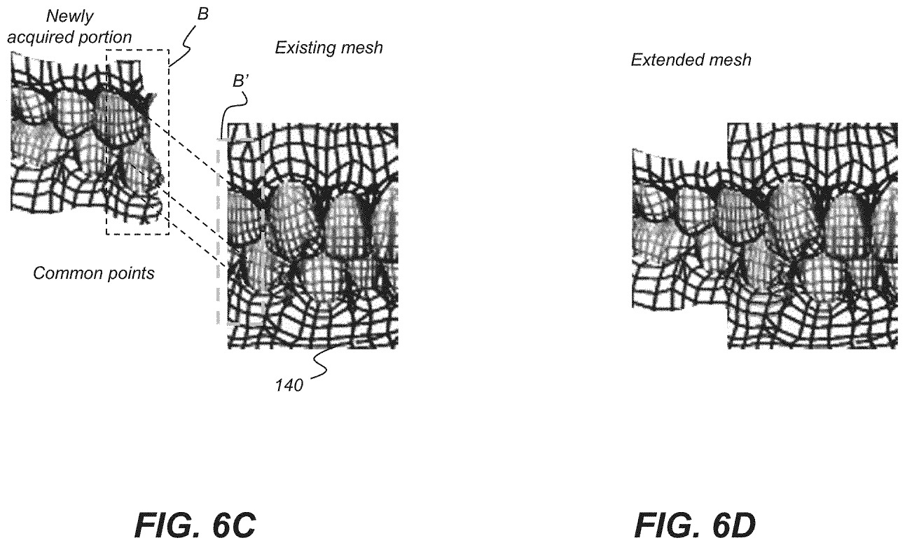

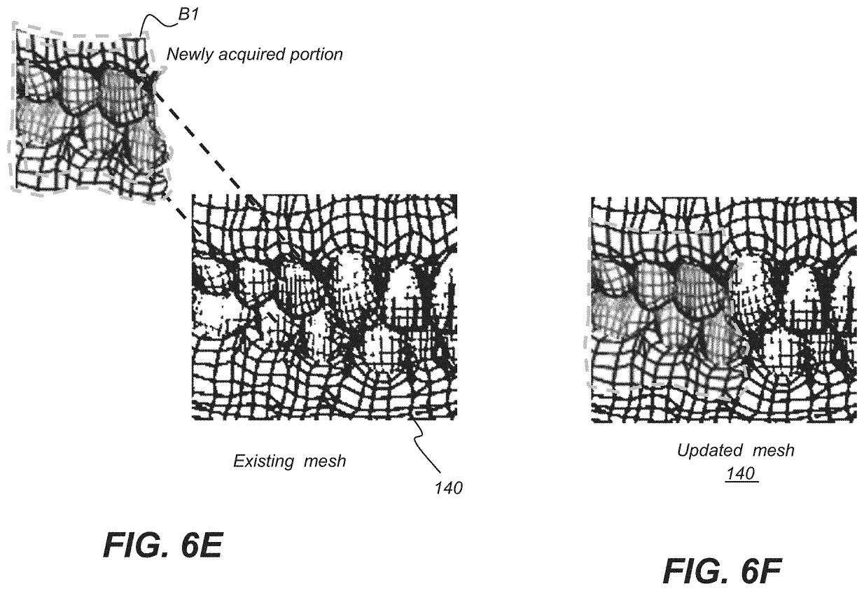

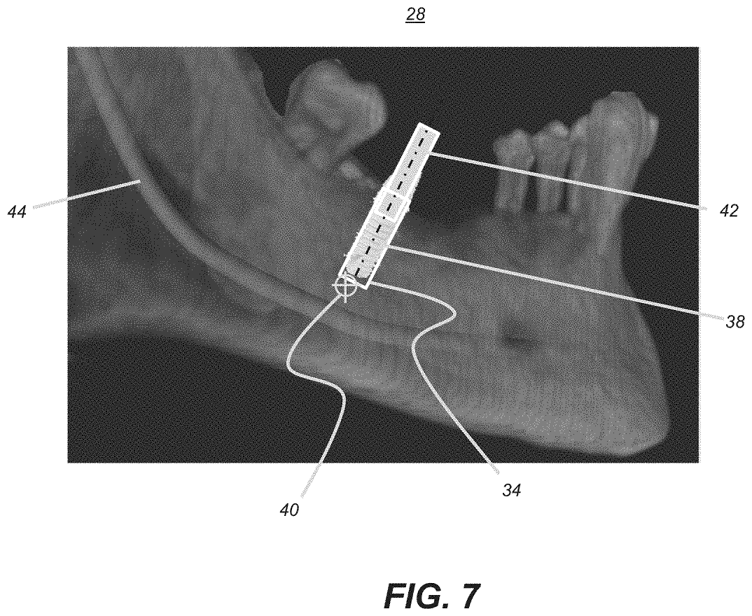

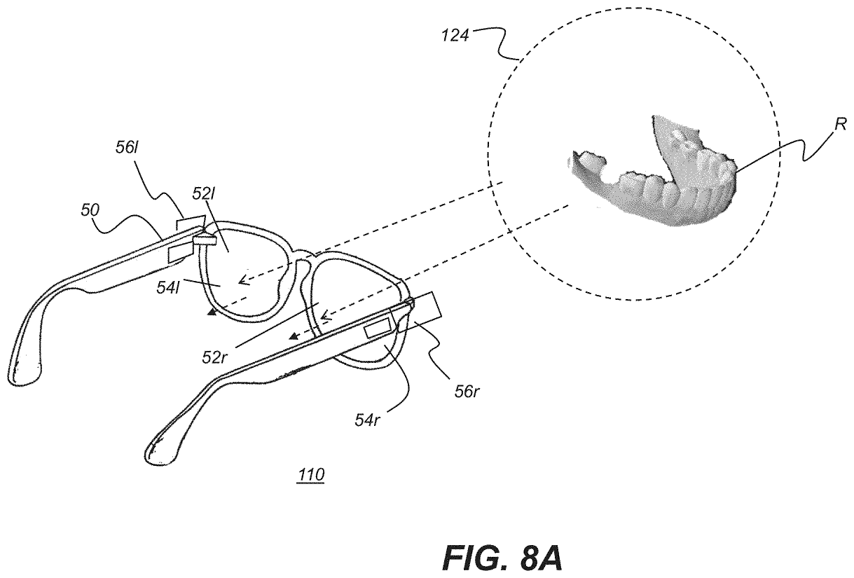

By way of example, FIG. 6A schematically shows overlaid structured light images 26a, 26b, and 26c obtained over a treatment region R. Each of structured light images 26a, 26b, and 26c can have projected line segments used for surface characterization as described previously with reference to FIGS. 3 and 4. The respective structured light images 26a, 26b, and 26c are slightly shifted in phase from each other to provide contour information over the treatment region R. Their combination can be used to provide the needed information to generate or update mesh 140 as shown in FIG. 5.

Embodiments of the present disclosure not only allow for updating of mesh 140, but also allow for its expansion according to structured light image data over areas adjacent to treatment region R. By way of example, FIG. 6B schematically shows overlaid structured light images 26a, 26b, and 26c obtained over a treatment region of dentition R, with added structured light images 27a, 27b, and 27c taken over adjacent region of dentition R1. Region R1 at least slightly overlaps treatment region R. By taking advantage of overlapped surface data and position information acquired from the imaging apparatus 70 (FIG. 2), control and processing logic on processor 80 can extend the surface contour information beyond its initial boundaries. This capability can be of particular value when it is useful to obtain surface contour information that includes a portion of a surgical instrument such as a dental drill, for example, that is working at a surgical site location along and beneath the surface of treatment region R, as described in more detail subsequently.

FIGS. 6C and 6D show how a newly acquired mesh portion 142 can be used to extend an existing mesh 140. A boundary region B of a newly acquired mesh portion 142 is identified and matched for overlap with the corresponding mesh content on existing mesh 140. Boundary or overlap region B includes area along the periphery of newly acquired mesh portion 142. As can be seen in FIG. 6C, boundary region B in newly acquired mesh portion 142 corresponds to boundary region B′, shown in dashed outline in existing mesh 140. In certain embodiments described herein, a shape of the boundary or overlap region B can also be used to determine the position of the intraoral scanner relative to the mesh.

Update of the existing mesh 140 can also be accomplished in a similar way to extension of the mesh. FIG. 6E shows how newly acquired mesh portion 142 can be used to update an existing mesh 140. Here, a boundary region B1 of a newly acquired mesh portion 142 is identified, shown between dashed outlines, and matched with the corresponding mesh content on existing mesh 140. In the update case, boundary region B1 includes area along each edge of the periphery of newly acquired mesh portion 142. FIG. 6F shows an updated mesh 140 that incorporates the newly scanned mesh content.

In certain exemplary embodiments, the existing mesh 140 can be updated when a newly acquired 3-D image (e.g., newly acquired 3-D image 142) partly overlaps with 3-D surface of the existing mesh 140 by augmenting the existing mesh 140 with a portion of the newly acquired 3-D image that does not overlap with the existing mesh 140. Further, when the newly acquired 3-D image completely overlaps with the existing mesh 140, existing mesh 140 can be updating in real time by replacing the corresponding portion of the existing mesh 140 with the contents of newly acquired 3-D image. In other words, complete overlap occurs when the newly acquired 3-D image falls within the boundaries of the existing mesh 140 or completely covers a portion of the existing mesh that is totally included within the boundaries of the existing mesh 140. In one embodiment, the corresponding portion of the existing mesh 140 that was replaced no longer contributes to the updated existing mesh 140.

In certain exemplary method and/or apparatus embodiments, determining a position of an intraoral scanner relative to the existing mesh 140 in real time can be performed by comparing the size and the shape of the overlap to the cross-section of the field-of-view of the intraoral scanner. Preferably, the size and the shape of the overlap of a newly acquired 3-D image is used to determine the distance and the angles from which the newly acquired 3-D image was acquired relative to the 3-D surface of the existing mesh 140.

In one exemplary embodiment, determining a position of an intraoral scanner relative to the existing mesh 140 in real time can be preferably performed when at least 50% of the newly acquired 3-D image overlaps the existing mesh 140. However, in certain exemplary method and/or apparatus embodiments, determining a position of an intraoral scanner relative to the existing mesh 140 in real time can be performed when 20%-100% of the newly acquired 3-D image overlaps the existing mesh 140. In some exemplary embodiment, determining a position of an intraoral scanner relative to the existing mesh 140 in real time can be performed when greater than 75% or greater than 90% of the newly acquired 3-D image overlaps the existing mesh 140.

The capability to generate, extend, and update the mesh 140 can be provided by a scanner that is coupled to the surgical instrument itself, as described in more detail subsequently. This arrangement enables real-time information to be acquired and related to the surgical site within the treatment area and/or position of the surgical instrument relative to the mesh and/or practitioner. Continuous tracking of this information enables visualization tools associated with the treatment system to display timely instructional information for the practitioner.

An embodiment of the present disclosure can be used for providing assistance according to a surgical treatment plan, such as an implant plan that has been developed using existing volume image content and a set of 2-D contour images of the patient. Implant planning, for example, uses image information in order to help locate the location of an implant fixture relative to nearby teeth and to structures in and around the jaw, including nerve, sinus, and other features. Software utilities for generating an implant plan or other type of surgical plan are known to those skilled in the surgical arts and have recognized value for helping to identify the position, dimensions, hole size and orientation, and overall geometry of an incision, implant, prosthetic device, or other surgical feature. Surgical treatment plans can be displayed as a reference to the practitioner during a procedure, such as on a separate display monitor that is viewable to the practitioner. However, conventional display approaches have a number of noteworthy limitations. Among problems with conventional surgical plan display is the need to focus somewhere other than on the patient; the practitioner must momentarily look away from the incision or drill site in order to view the referenced surgical plan. Additionally, the plan is not updated once the procedure begins, so that displayed information can be increasingly less accurate, such as where surface material is removed or moved aside. An embodiment of the present disclosure addresses these problems by providing surgical plan data, continuously updated, using ongoing surface scanning as well as augmented reality display tools. An embodiment of the present disclosure can provide surgical plan data, continuously updated, using ongoing surface scanning as well as augmented reality display tools registered to the field of view of the practitioner.

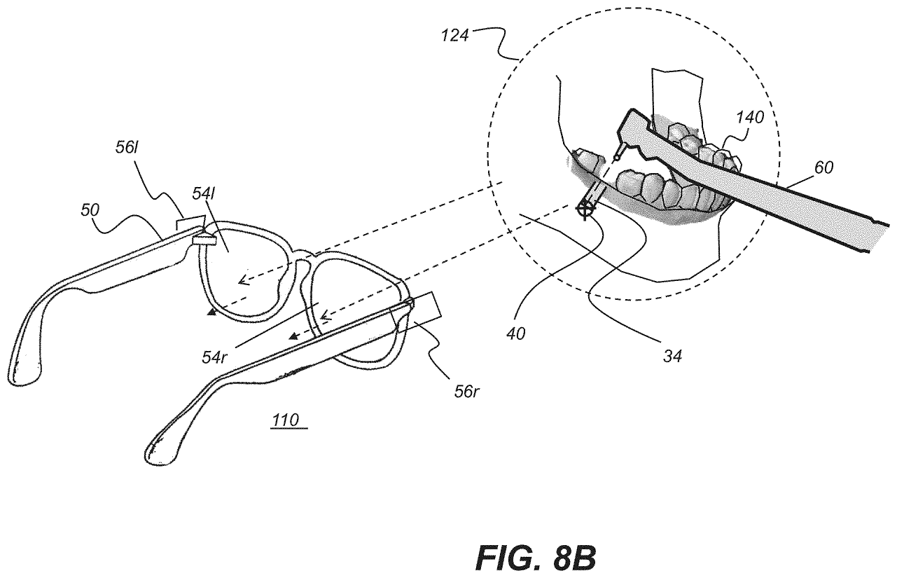

FIG. 7 shows an image 28 generated using surgical planning utilities such as for an implant plan. The implant plan can generate a figure of this type, showing location of a hole 34 for an implant 38 and a corresponding drill path 42 and target 40 as an end-point for the drilling process. A nerve 44 is also displayed.

The implant plan can initially use 3-D information from both volumetric imaging, such as from a CBCT apparatus, and surface contour imaging, such as from a structured light scanning device. The two sets of data, volumetric and surface contour, relative to each other and the initial implant plan, can give the practitioner useful information related to both visible surfaces and invisible tissue beneath the surface. Advantageously, as execution of the plan progresses, embodiments of the present disclosure allow recomputation and updating of the displayed surface, based on work performed by the practitioner.

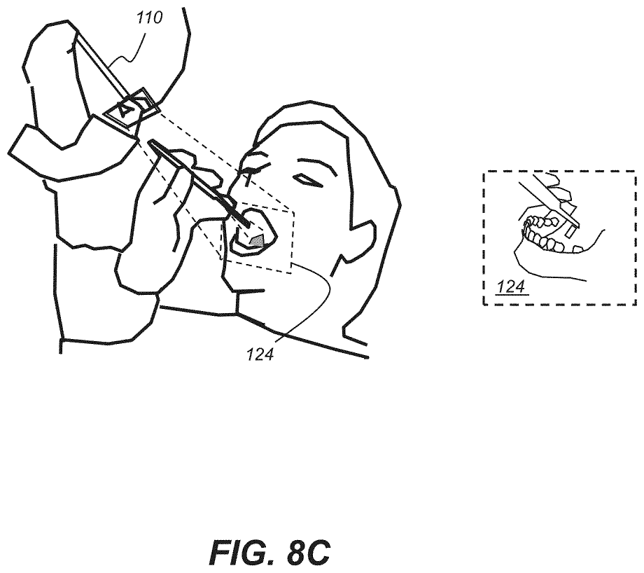

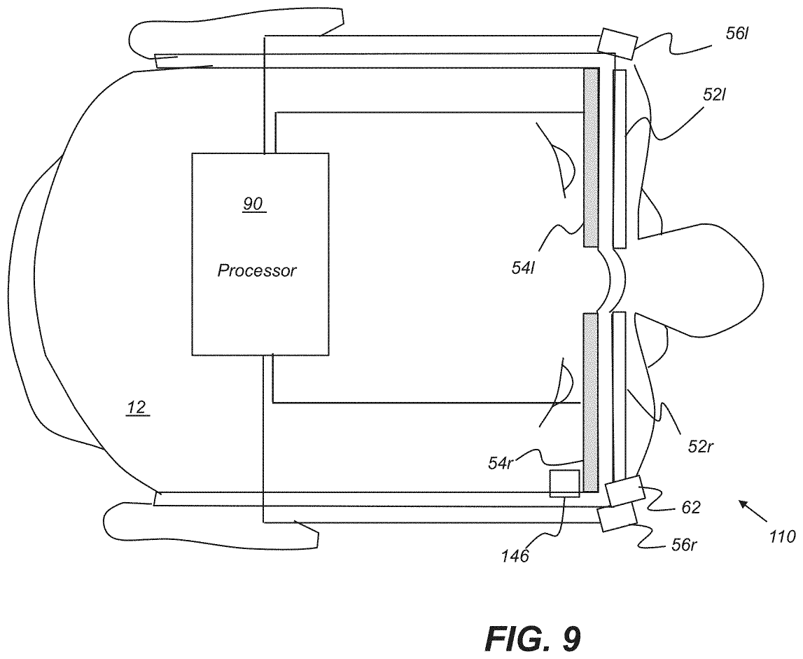

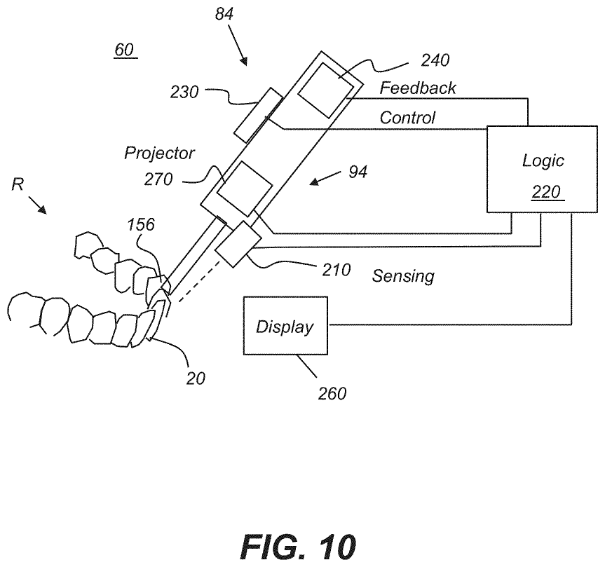

The schematic view of FIG. 8A shows head-mounted device (HMD) 110 as worn by a practitioner according to an embodiment of the present disclosure. A field of view (FOV) 124 is visible to the practitioner through a left lens 52l and a right lens 52r, provided by HMD 110, and includes at least treatment region R of the patient. For augmented reality display, left- and right-eye display elements 541 and 54r form an image visible to the practitioner, such as a stereoscopic image, for example; however, the display content can be superimposed on the field of view of the practitioner, without blocking visibility of the patient's teeth or other viewed structures. As the schematic view of FIG. 8B shows, the display content can include features of the surgical plan, such as hole 34 and target 40, as well as a generated display of a surgical instrument 60 and surface contour image data, such as mesh 140 overlaid onto or combined with surgical plan image contents. The combined surface contour and volume image content can be continually refreshed, along with displayed information related to instrument 60 positioning, to provide the viewing practitioner with updated, real-time surgical plan information, all displayed within field of view 124 of the practitioner. Using this utility, the practitioner can keep eyes focused on the surgical procedure without interrupting the continuous view of the patient.

FIG. 8C shows how head-mounted device 110 can define field of view 124 for the practitioner. HMD 110 is capable of providing synthetic virtual image content that can be at least partially transparent, so that a field of view can be defined that includes both real-world content and virtual image content generated by a computer and intended to provide surgical guidance.

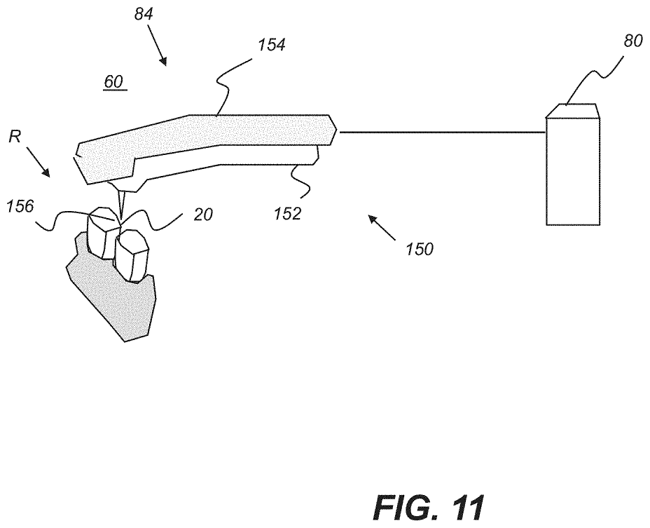

The schematic diagram of FIG. 9 shows various components of HMD 110 for augmented reality viewing. HMD 110 is in the form of eyeglasses or goggles worn by a practitioner 12. HMD 110 has a pair of transparent lenses 52l and 52r for left and right eye viewing, respectively. Lenses 52l and 52r can be corrective lenses, such as standard prescription lenses specified for the practitioner, or can be plano lenses. HMD 110 also has a pair of left and right display elements 541 and 54r, such as planar waveguides for providing computer-generated stereoscopic left-eye and right-eye images, respectively. Display elements 541 and 54r can be incorporated into lenses 52l and 52r, such as using waveguides with diffractive input and output sections, for example. Planar waveguides that provide this function are described, for example, in U.S. Patent Application Publication No. 2010/0284085 by Laakonen.

Continuing with the FIG. 9 description, a processor 90, which may be a dedicated logic processor, a computer, a workstation, or combination of these types of devices or one or more other types of control logic processing device, provides the computer-generated image data to display elements 541 and 54r. A pair of cameras 56l and 56r are mounted on HMD 110 for recording at least the field of view of the practitioner. A single camera could alternately be used for this purpose. These images go to processor 90 for image processing and position detection, as described in more detail subsequently. Additional optional devices may also be provided with HMD 110, such as position and angle detection sensors, audio speakers, microphone, or auxiliary light source, for example. An optional camera 146 can be used to detect eye movement of practitioner 12, such as for gaze tracking that can be used to determine where the practitioner's attention is directed. In one embodiment, gaze tracking can help to provide information that is compatible with the attention and area of interest of the practitioner. An optional projector 62 can be provided for projecting a beam of light, such as a scanned beam or a modulated flat field of light, as illumination for portions of the tooth or other structure of interest to the practitioner. Projected light can have different colors indicating different types of material in the field of view, such as bone and restoration material. This can help the practitioner to distinguish optically similar materials.

HMD devices and related wearable devices that have cameras, sensors, and other integrated components are known in the art and are described, for example, in U.S. Pat. No. 6,091,546 to Spitzer et al.; U.S. Pat. No. 8,582,209 to Amirparviz; U.S. Pat. No. 8,576,276 to Bar-Zeev et al.; and in U.S. Patent Application Publication 2013/0038510 to Brin et al. HMD devices are capable of superimposing image content onto the field of view of the wearer, so that virtual or computer-generated image content appears to the viewer along with the real-world object that lies in the field of view, such as a tooth or other anatomy.

For the superimposition of computer-generated image features as virtual images from the surgical plan onto the real-world view of the patient's mouth in field of view 124 (FIG. 8B), the computer-generated image content, such as target 40 in FIG. 8B, can be positionally registered with the view that is detected by cameras 56l and 56r in FIG. 9. Registration with the field of view can be performed in a number of ways; methods for registration of a computer-generated image to its real-world counterpart are known to those skilled in the arts, including the use of object and shape recognition for teeth or other features, for example. Registration techniques for visualization can employ conventional techniques used in registration for preparing surgical guides, for example.

Registration of mesh content with the field of view can be performed by the apparatus shown in FIG. 9 in which cameras 56l and 56r record images of the FOV and provide this image data to processor 90. As the FOV can be constantly changing during a treatment session, recomputation of the FOV from images obtained allows the display apparatus to change superimposed imaging content and/or registered superimposed imaging content accordingly. Head movement by the practitioner, for example, can require the display apparatus to change the angle at which content is viewed.

According to an embodiment of the present disclosure, a registration sequence is provided, in which the practitioner follows initial procedural instructions for setting up registration coordinates, such as to scan the region of interest using an intra-oral camera 24 (FIG. 2) or to view the patient from a specified angle to allow registration software to detect features of the patient anatomy. According to an alternate embodiment of the present disclosure, image feature recognition software is used to detect features of the face and mouth of the patient that help to correlate the visual field to the volume image data so that superposition of the virtual and real images in the field of view (FOV) is achieved. Image feature recognition software algorithms are well known to those skilled in the image processing arts. According to an embodiment of the present disclosure, feature recognition software processing uses stored patient image data and is also used to verify patient identification so that the correct information for the particular patient is shown.

Progress indicators can be provided by highlighting a particular tooth or treatment area of the mouth or other anatomy by the display of overlaid image content generated from processor 90 (FIG. 9). Visual progress indicators can include displayed elements that appear in the background or along edges of the displayed content. Colors or flashing of the overlaid image can be provided in the augmented reality display in order to indicate the relative status of a treatment or procedure.

According to an embodiment of the present disclosure, progress indicators are provided by overlaid virtual images according to system tracking of treatment progress at the surgical site. For drilling a tooth, image content can show the practitioner features such as drill location, drill axis, depth still needed according to the surgical plan, and completed depth thus far, for example. As the drill nears the required depth, image content can be changed to reflect the treatment status and thus help to prevent the practitioner from drilling too deeply. Display color can be used, for example, to indicate when drilling is near-complete or complete. Display color can also be used to indicate proper angle of approach or drill axis and to indicate whether or not the current drill angular position is suitably aligned with the intended axis or should be adjusted.

According to an embodiment, image content is superimposed on the practitioner FOV only when treatment thresholds or limits are reached, such as when a drilled hole is at the target depth or when the angle of a drill or other instrument is incorrect. In one embodiment, deviation information to the practitioner can be registered onto the field of view and oriented to the field of view when the sensed position of a surgical instrument is contrary to the surgical treatment plan. Exemplary deviation information is a representation (e.g., orientation) of the surgical instrument and correction information in accordance with the surgical treatment plan displayed in the practitioners' field of view registered to the actual object as seen from the practitioners' field of view. With continual monitoring of the surgical site by the camera that is coupled with the surgical instrument, up-to-date information is available on treatment progress and can be refreshed continually so that treatment status can be reported with accuracy.

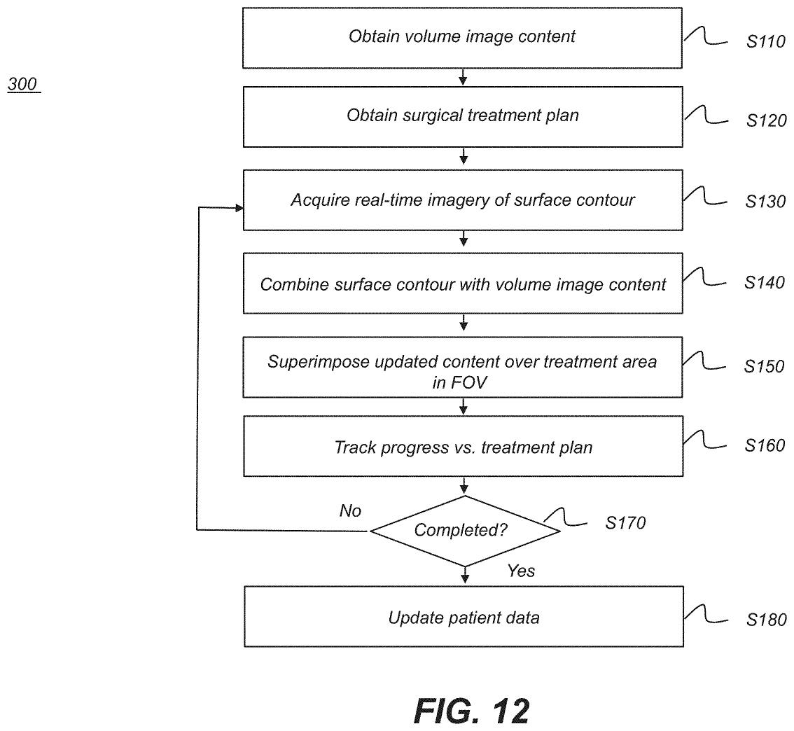

Real-time images from treatment region R in the practitioners FOV can be obtained from a camera and from one or more image sensors provided in a number of different ways. FIG. 9 showed how images can be acquired using HMD 110, for real-time display to the practitioner. Images of the treatment area can also be acquired from a camera provided on a dental instrument, for example. The schematic diagram of FIG. 10 shows instrument 60 that includes sensing circuitry 210 that may include a camera or image sensing device, for example. In an exemplary embodiment, sensing circuitry 210 may include projection and detection components that form an introral scanner 94 that is coupled to instrument 60 for providing structured light images of the surgical instrument 60, such as a drill tip, as well as of a portion of the treatment area for example. Projector 270 can be used to project a structured light pattern or other useful pattern onto surface 20 for contour imaging. Instrument 60 may acquire images during use or at particular intervals between actuations. A control logic processor 220 coordinates and controls the processing of signals obtained from sensing circuitry 210, such as a camera or other imaging device, and cooperates with control circuitry 230 and settings made by the practitioner for using instrument 60. Control circuitry 230 can also actuate instrument 60 to perform various functions and report on progress through sensing circuitry 210. Feedback circuitry 240 provides one or more feedback signals that are used by control logic processor 220 to control and provide information about procedures underway using instrument 60. Control circuitry 230 can also be coupled to a display 260 (e.g., of a workstation, computer or the like) for concurrent display of acquired image content, feedback signals and/or for subsequent post-acquisition review, processing and analysis of acquired image content.

Other possible types of sensors that can be used to indicate instrument location or orientation include optical sensors, including sensors that employ lasers, and ultrasound sensors, as well as a range of mechanical, Hall effect, and other sensor types.

It has been noted that structured light imaging is only one of a number of methods for obtaining and updating surface contour information for intraoral features. Other methods that can be used include multi-view imaging techniques that obtain 3-D structural information from 2-D images of a subject, taken at different angles about the subject. Processing for multi-view imaging can employ a “structure-from-motion” (SFM) imaging technique, a range imaging method that is familiar to those skilled in the image processing arts. Multi-view imaging and some applicable structure-from-motion techniques are described, for example, in U.S. Patent Application Publication No. 2012/0242794 entitled “Producing 3D images from captured 2D video” by Park et al., incorporated herein in its entirety by reference. Other methods for characterizing the surface contour use focus or triangularization of surface features, such as by obtaining and comparing images taken at the same time from two different cameras at different angles relative to the subject treatment region.

Force monitoring can be applied to help indicate how much force should be applied, such as in order to extract a particular tooth, given information obtained through images of the tooth. Force monitoring can also help to track progress throughout the procedure. Sensing can be provided to help indicate when the practitioner should stop or change direction of an instrument, or when to stop to avoid other structures. Excessive force application can also be sensed and can cause the system to alert the practitioner to a potential problem. The system can exercise further control by monitoring and changing the status or speed of various tools according to detected parameters. Drill speed can be adjusted for various conditions or the drill or other instrument slowed or stopped according to status sensing and progress reporting. Radio-frequency (RF) sensing devices can also be used to help guide the orientation, positioning, and application of surgical and other instruments.

According to an embodiment of the present disclosure, the tool head of a drill or other surgical instrument 60 can be automatically swapped or otherwise moved in order to allow imaging of a surface 20 or element being treated. A telescopic extension can be provided to help limit or define the extent of depth or motion of a tool or instrument.

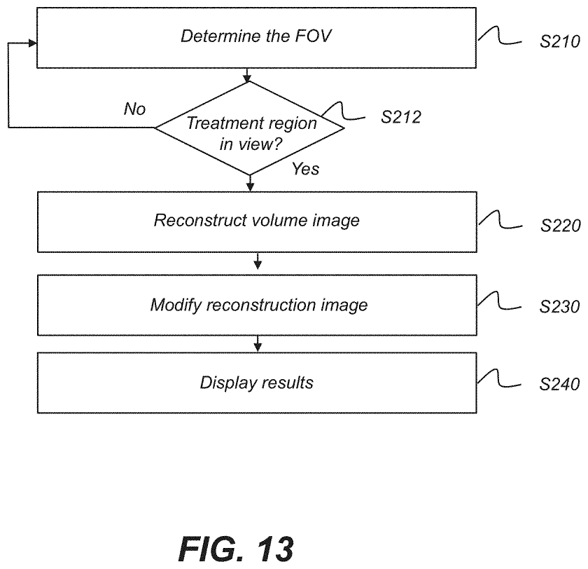

According to an alternate embodiment of the present disclosure, as shown in surgical instrument 60 of FIG. 10 and in surgical instrument 150 of FIG. 11, dental drill 152 or other instrument type is coupled to intra-oral imaging camera 154 or other sensing circuitry 210 as part of an intra-oral scanner 84 that is coupled to a dental treatment instrument 60. Scanner 84 includes camera 154 with light source that provides structured light illumination that supports contour imaging (not shown in FIG. 11). Using dental instrument 60 having this configuration, a practitioner can have the advantage of imaging update during treatment activity, rather than requiring the camera 154 to pause in imaging while the practitioner drills or performs some other type of procedure at surgical site 156. Where mechanical coupling is used, scanner 84 clips onto drill 152 or other type of instrument 60, allowing the scanner to be an optional accessory for use where it is advantageous for characterizing surfaces of the treatment region R and its surgical site 156, and otherwise removable from the treatment tool. Camera 154 and associated scanner 84 components can similarly be clipped to other types of dental instruments, such as probes, for example.

Camera 154 and associated scanner 84 components can also be integrally designed into the drill or other instrument 150, so that it is an integral part of the dental instrument 150. Camera 154 can be separately energized from the dental instrument 150 so that image capture takes place with appropriate timing. Exemplary types of dental instruments 150 for coupling with camera 154 and associated scanner 84 components can include drills, probes, inspection devices, polishing devices, excavators, scalers, fastening devices, and plugging devices.

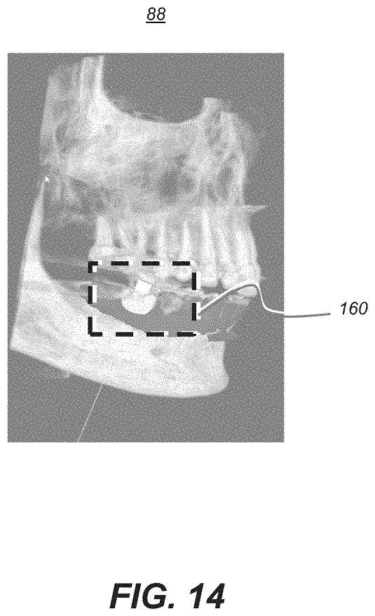

FIG. 12 is a logic flow diagram that shows a sequence of steps used in an embodiment with the general workflow of surgical guidance and tracking functions provided by imaging system 100 of FIG. 1. In a workflow sequence 300, a volume image content acquisition step S110 acquires the processed CBCT scan data or other image data that can be used for reconstruction of a volume image that includes voxel values for tissue that is on the surface as well as beneath the surface of the dental or other anatomy feature. An obtain surgical treatment plan step S120 then obtains the surgical treatment plan developed using the acquired volume image content for the patient. A contour image acquisition step S130 executes, in which structured light images that include the treatment region and surgical site are obtained, such as from a scanning apparatus that is coupled to the surgical instrument or from scans provided from illumination and camera on an HMD or other image source. The structured light images are processed in order to provide contour image data. Alternately, other types of image content can be used in order to provide characterization of the treatment region surface. Iterative processing follows, during which an image combination step S140 combines image content of the treatment region from the volume image content and from the most recently acquired contour image content obtained from the surgical site. This combination forms a 3-D or volume virtual model that can then be combined with surgical treatment data to form an example of a surgical treatment plan for the patient. In a display step S150, the practitioner's field of view is acquired and the combined image from step S140 is used to superimpose features from the surgical treatment plan relative to or registered to corresponding features in the FOV. Optionally, step S150 also prompts the practitioner for the process of carrying out the identified surgical treatment procedure. A tracking step S160 tracks procedure progress relative to the surgical treatment plan, measuring and reporting on the procedure and position of the surgical instrument as it is used at the surgical site. Tracking step S160 and a test step S170 then initiate iteration of the contour image acquisition and image combination steps S130 and S140 in an ongoing manner, updating the display in step S150 with each iteration as execution of the treatment proceeds. An update step S180 then updates stored patient data according to the procedure executed and images obtained. The superimposed image content can be stored, displayed, or transmitted, such as to provide a visual record of the surgical procedure.

It should be noted that step S110 of FIG. 12 can be optional, so that the surgical plan provides only information relative to surface structures and does not require a volume imaging system, such as a CBCT apparatus, for example. In such a case, only surface contour data is obtained and processed.

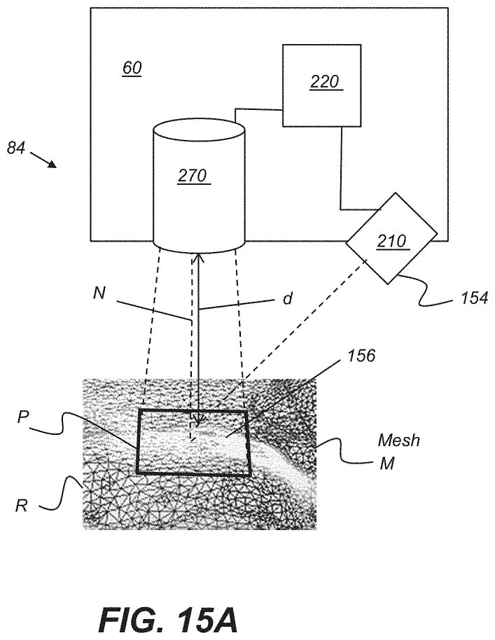

According to an embodiment of the present disclosure, as shown in the logic flow diagram of FIG. 13, combination of the contour imaging data with the volume image content for a given FOV is a process of:
(i) Determining the FOV based on camera information from the head-mounted device in a FOV determination step S210. Then, in an FOV analysis step S212, determining whether or not the treatment region lies within the FOV. If not, activity returns to the FOV determination step S210 until the practitioner FOV includes the treatment region.
(ii) Reconstructing the volume image data to provide a 3-D view or, alternately, to generate image slices according to the FOV in a reconstruction step S220.
(iii) Modifying the reconstruction according to contour imaging data in a modification step S230. This can include, for example, making a subset of the image voxels transparent, such as where a feature has been removed or a hole drilled.
(iv) Displaying results in a display step S240.

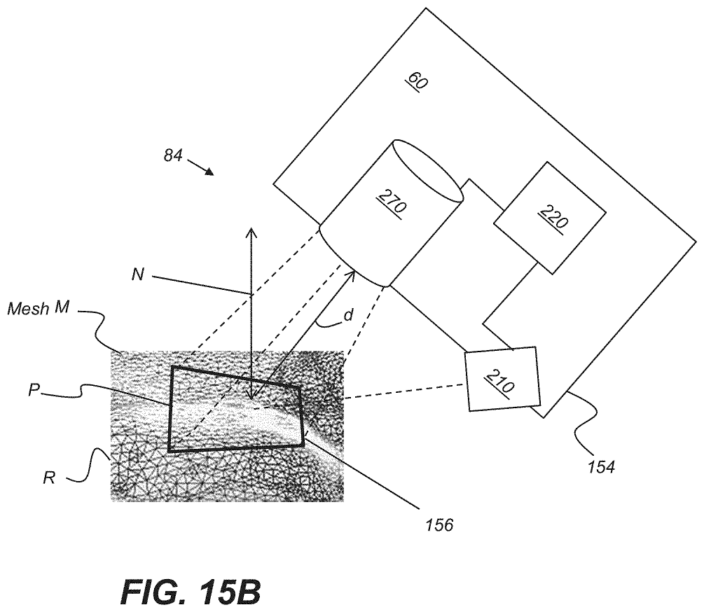

FIG. 14 shows an exemplary display view of an image 88 for guidance in a dental procedure. In the example shown, head-mounted device 110 provides an image of a crown position 160 and related teeth of the lower jaw, superimposed over the visual field of the dental practitioner.

### Real-Time Instrument Location and Surface Status

According to an aspect of the present embodiment, surgical instrument 60 (FIG. 10) has the capability to update volume image content in real-time, allowing the practitioner to have ongoing visual feedback that supports a surgical procedure. As a treatment proceeds, the updated display on the HMD of the practitioner shows real time changes to the treatment region (e.g., image content superimposed and/or registered to the actual object and presented in the detected practitioner's field of view) and can provide status information and/or deviation information on progress relative to the surgical plan. The status information can be alphanumeric, symbolic, or any suitable combination of synthetic information generated by the computer to support a surgical treatment.

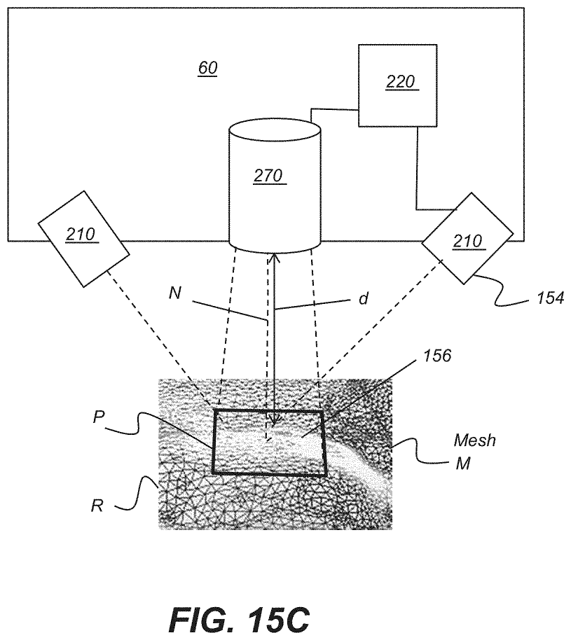

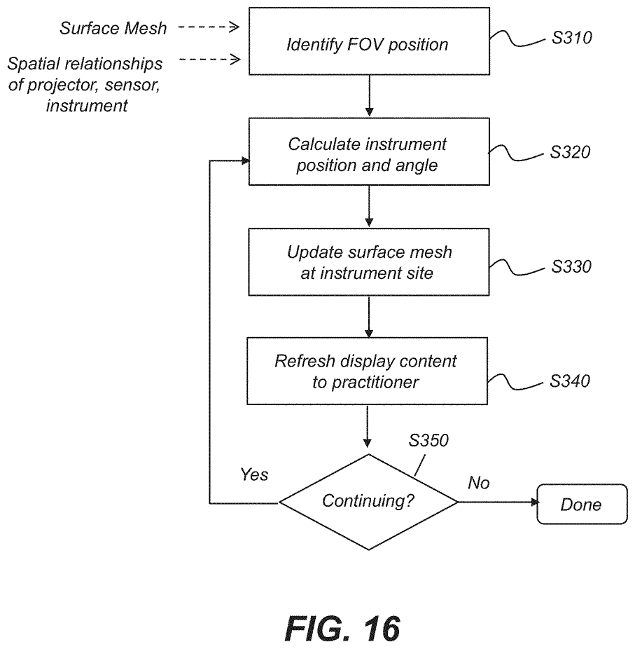

The schematic views of FIGS. 15A and 15B show how surgical instrument 60 can identify its position relative to a surgical instrument site 156 in a treatment region R and can provide updated image information related to changes in the treatment region of the patient according to the surgical plan. Image sensing circuitry 210 is provided by camera 154 of intra-oral scanner 84 that is coupled to instrument 60 control logic. The camera of sensing circuit 210 provides ongoing image capture and processing in order to generate and update mesh M. In certain exemplary embodiments, the mesh M can be updated in real time when a newly acquired 3-D contour image partly overlaps with 3-D surface of the mesh M by adding a portion of the newly acquired 3-D contour image that does not overlap with the mesh M to the mesh M. Further, the existing mesh M can be updating in real time by replacing the corresponding portion of the existing mesh M with the contents of newly acquired 3-D contour image that completely overlaps with the existing mesh M. In one embodiment, the corresponding portion of the existing mesh M that was replaced no longer contributes to the updated existing mesh and/or is stored for later use or discarded.

Projector 270 of scanner 84 directs a pattern P of light of a prescribed shape onto the surface of the treatment region R. In certain embodiments, determining a position of an intra-oral scanner 84 relative to the existing mesh M in real time can be performed by comparing the size and the shape of the overlap on the mesh M to the cross-section of the field-of-view of the intraoral scanner. Preferably, the size and the shape of the overlap (e.g., position of the projected light pattern P on the mesh M) of a newly acquired 3-D contour image is used to determine the distance and the angles from which the newly acquired 3-D contour image was acquired relative to the 3-D surface of the existing mesh M. In an alternative embodiment, combined information about relative distortion or deformation of size and shape of the projected pattern P of light and the detected surface contour of the mesh M within pattern P allow calculation of distance d between projector 270 and the surface and calculation of the angle of instrument 60 relative to a normal N to a reference point on the surface or other angular reference. For example, the outline of projected pattern P is distorted according to the deviation of projector 270 angle from normal, as well as according to the varying slope and contour of the surface. For example, the light beam that forms projected pattern P can have a rectangular or circular cross-section as output from projector 270. However, the distortion of the pattern P outline on the surface can be used to compute distance and angle that indicates the position of intra-oral scanner 84, taking into account the slope and features of the imaged surface.

The schematic view of FIG. 15C shows an alternate embodiment for surgical instrument 60 having two sensing circuits 210 to detect the shape of pattern of light P using triangulation. Feature identification can alternately be used to detect the relative angle of the surgical instrument 60 using its scanner apparatus. In addition, deformation of features or deformation apparent in the FOV itself can be used to identify intra-oral scanner location.

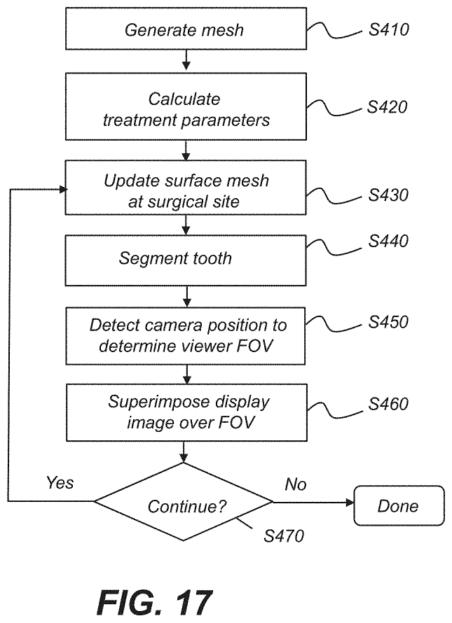

The logic flow diagram of FIG. 16 shows a sequence for detection of instrument 60 position using the arrangement described with reference to FIGS. 10, 11, and 15. An FOV determination step S310 identifies the field of view based on surface mesh data previously obtained as well as image data currently being obtained by the camera that is coupled to the instrument. FOV determination step S310 can also use known spatial and angular relationships of the instrument, including relative positions and inclinations of projector and sensor components. A calculation step S320 obtains this mesh and positional data and calculates instrument position and angle accordingly. This calculation includes shape of the projected pattern P, as previously described with reference to FIGS. 15A and 15B. A mesh update step S330 then updates the local mesh information obtained from images of the surgical instrument site. The mesh update can include updating the volume image content, including information obtained from both reflectance images and radiographic images. As one example, where the instrument is a dental drill, mesh update step S330 determines where the drill has changed the surface contour and updates mesh data accordingly. A refresh step S340 refreshes the display content for the practitioner based on the localized mesh recomputation. A test step S350 determines whether or not to repeat calculation, update, and refresh procedures of preceding steps, such as when the drill is still operating or based on other detection.

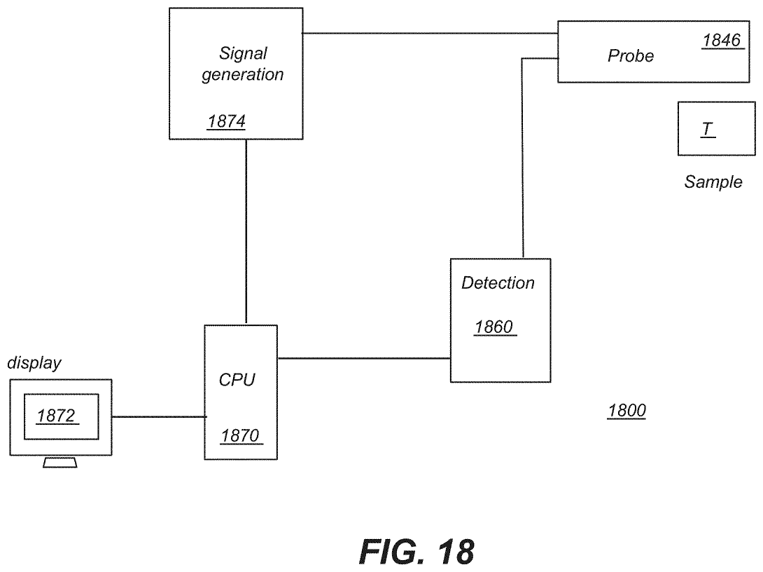

The logic flow diagram of FIG. 17 shows a sequence for providing display content that supports a dental surgical procedure. A mesh generation step S410 forms a 3-D mesh according to a surface contour of a patient's mouth and including a treatment area. A treatment parameters calculation step S420 then calculates treatment parameters for the dental procedure, based on the mouth anatomy of the patient. The treatment parameters can include implant shape and margin line definition, restoration shape information, and other data that relate to the intended procedure and will be used to guide the practitioner in subsequent steps. A mesh update step S430 can then be executed. Mesh update step S430 uses image data obtained from a camera that is part of an intra-oral scanner coupled to the surgical instrument, as described previously. As surgery proceeds, the camera acquires reflectance images that show changes to the tooth structure at the surgical site, such as the drilling site for example. A segmentation step S440 can then execute to segment the tooth of interest for the surgical procedure. A FOV determination step S450 then detects the position of a second camera that is coupled to the practitioner, such as a camera that is part of an HMD, as described previously. The head-mounted camera obtains image content that can be used to detect the position of the practitioner relative to the segmented tooth. A display step S460 is executed, in which data from the calculated treatment parameters, conditioned by the updated mesh information from step S430, is displayed superimposed over the practitioner's field of view, such as using the HMD device. In one embodiment, at least some of the updated mesh information is registered (e.g., to actual object) in the detected practitioners' field of view. A test step S470 then determines whether or not the procedure is complete or should be continued, either of which can be displayed to the practitioner.

In certain exemplary method and/or apparatus embodiments, for updating display of a dentition to a practitioner, first 3-D surface contour image content such as a 3-D mesh and/or radiographic volume image content such as a 3-D volume reconstruction that includes a dentition treatment region can be obtained. Then, the 3-D surface contour image content and the radiographic volume image content can be combined into a single 3-D virtual model that includes the dentition treatment region. Next, the practitioner's field of view can be detected and at least a portion of the single 3-D virtual model can be display preferably superimposed and oriented to the practitioner's field of view to be registered to the actual dentition treatment region as seen from the practitioner's field of view. Next or concurrently to the previous steps, a surgical treatment plan related to the dentition treatment region can be obtained and preferably displayed by corresponding virtual image data in the practitioner's field of view.

Then repeatedly, and preferably in real time, the 3-D surface of the dentition treatment region is updated by replacing the corresponding portion of the 3-D surface of the dentition treatment region with contents of newly acquired 3-D images of the dentition treatment region that comprise physical dental objects in the dentition treatment region from different points of view using a 3-D intra-oral scanning device. In one embodiment, the replaced corresponding portion of the 3-D surface of the dentition no longer contributes. Concurrently, the position of a surgical instrument, preferably mounted to the 3-D intra-oral scanning device, is determined and can be displayed, for example by corresponding virtual image data in the practitioner's field of view, relative to the single 3-D virtual model. Also, concurrently, the superimposed single 3-D virtual model can be updated and continuously or intermittently displayed at the practitioner's field of view registered to actual objects in the dentition treatment region as seen from the practitioners' field of view according to the surgical treatment plan.

Further, deviation information can be provided to the practitioner superimposed onto the practitioner's field of view by corresponding virtual image data oriented to the field of view when the sensed position of a surgical instrument is contrary to the surgical treatment plan. In one embodiment, the deviation information can be an orientation of the surgical instrument and correction information in accordance with the surgical treatment plan displayed in the practitioners' field of view registered to the actual dentition treatment region as seen from the practitioners' field of view.

Additional deviation information can be for additional guided dental surgery related information and treatment plans. For example, the deviation information can include information related to and/or necessary to guide a surgical dental instrument to an entrance to a root canal of a selected tooth, information related to and/or necessary to excavate the root canal such as position, angle and orientation of the surgical dental instrument. Additional deviation information can be related to additional dental practice areas including endodontics or restorations.

In the context of the present disclosure, the term “camera” relates to a device that is enabled to acquire a reflectance, 2D digital image from reflected visible or NIR (near-infrared) light, such as structured light that is reflected from the surface of teeth and supporting structures.

Exemplary method and/or apparatus embodiments of the present disclosure provide a depth-resolved volume imaging for obtaining signals that characterize the surfaces of teeth, gum tissue, and other intraoral features where saliva, blood, or other fluids may be present. Depth-resolved imaging techniques are capable of mapping surfaces as well as subsurface structures up to a certain depth. Using certain exemplary method and/or apparatus embodiments of the present disclosure can provide the capability to identify fluid within a sample, such as saliva on and near tooth surfaces, and to compensate for fluid presence and reduce or eliminate distortion that could otherwise corrupt surface reconstruction.

Descriptions of the present invention will be given in terms of an optical coherence tomography imaging system. The invention can also be implemented using photo-acoustic or ultrasound imaging systems. For more detailed information on photo-acoustic and ultrasound imaging, reference is made to Chapter 7 “Handheld Probe-Based Dual Mode Ultrasound/Photoacoustics for Biomedical Imaging” by Mithun Kuniyil, Ajith Singh, Wiendelt Steenbergen, and Srirang Manohar, in Frontiers in Biophotonics for Translational Medicine”, pp. 209-247. Reference is also made to an article by Minghua Xu and Lihong V. Wang, entitled “Photoacoustic imaging in biomedicine”, Review of Scientific Instruments 77, (2006) pp. 041101-1 to -21.

### Imaging Apparatus

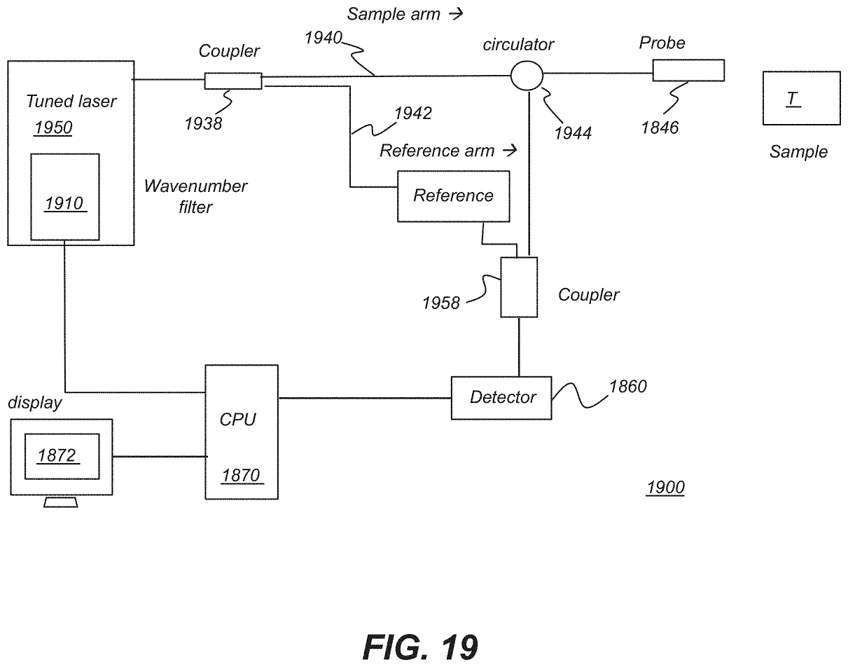

FIG. 18 shows a simplified schematic view of a depth-resolved imaging apparatus 1800 for intraoral imaging. Under control of a central processing unit, CPU 1870, and signal generation logic 1874 and associated support circuitry, a probe 1846 directs an excitation signal into the tooth or other intraoral feature, shown as a sample T in FIG. 18 and subsequent figures. Probe 1846 can be hand-held or fixed in place inside the mouth. Probe 1846 obtains a depth-resolved response signal, such as reflection and scattered signal, emanating from the tooth, wherein the response signal encodes structure information for the sampled tissue. The response signal goes to a detector 1860, which provides circuitry and supporting logic for extracting and using the encoded information. CPU 1870 then performs reconstruction of a 3D or volume image of the tooth surface or surface of a related feature according to the depth-resolved response signal. CPU 1870 also performs segmentation processing for identifying any fluid collected on or near the sample T and to remove this fluid from the 3D surface computation. A display 1872 then allows rendering of the 3D surface image content, such as showing individual slices of the reconstructed volume image. Storage and transmittal of the computed surface data or of an image showing all or only a portion of the surface data can also be performed as needed.

Following the basic model of FIG. 18, various types of signal generation logic 1874 can be used to provide different types of excitation signal through probe 1846. Among the excitation signal types that can be used are the following:
(i) OCT (optical coherence tomography), using a broadband light signal for time-domain, spectral, or swept-source imaging, as described in more detail subsequently;
(ii) ultrasound imaging, using an acoustic signal;
(iii) pulsed or modulated laser excitation, used for photo-acoustics imaging.

Depending on the type of excitation and response signals, accordingly, detection circuitry 1860 processes light signal for OCT or acoustic signal for ultrasound and photo-acoustic imaging.

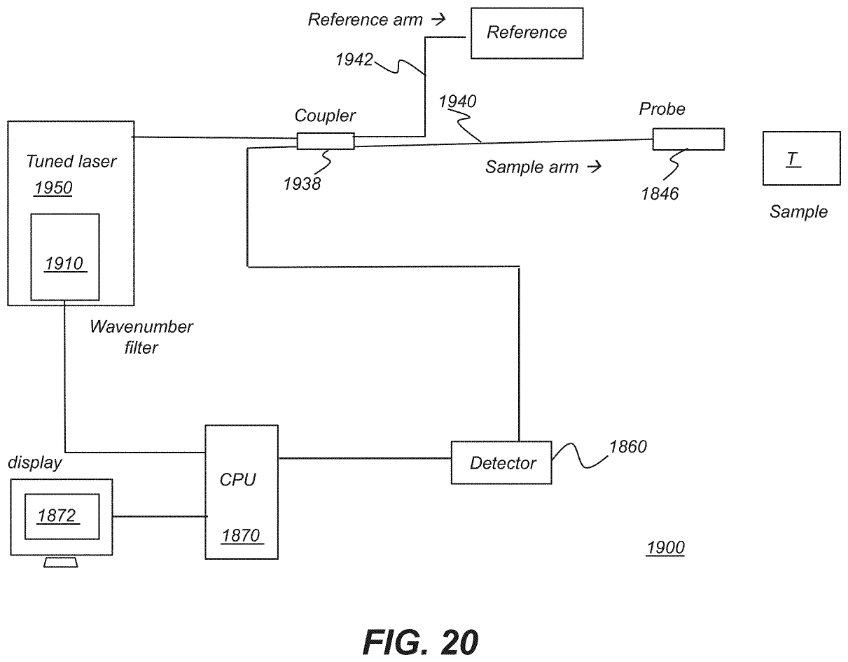

The simplified schematic diagrams of FIGS. 19 and 20 each show a swept-source OCT (SS-OCT) apparatus 1900 using a programmable filter 1910 according to an embodiment of the present disclosure. In each case, programmable filter 1910 is used as part of a tuned laser 50 that provides an illumination source. For intraoral OCT, for example, laser 50 can be tunable over a range of frequencies (wave-numbers k) corresponding to wavelengths between about 400 and 1600 nm. According to an embodiment of the present disclosure, a tunable range of 35 nm bandwidth centered about 830 nm is used for intraoral OCT.

In the FIG. 18 embodiment, a Mach-Zehnder interferometer system for OCT scanning is shown. FIG. 19 shows components for an alternate Michelson interferometer system. For these embodiments, programmable filter 1910 provides part of the laser cavity to generate a tuned laser 50 output. The variable laser 50 output goes through a coupler 1938 and to a sample arm 1940 and a reference arm 1942. In FIG. 18, the sample arm 1940 signal goes through a circulator 1944 and to a probe 1846 for measurement of a sample T. The sampled depth-resolved signal is directed back through circulator 1944 (FIG. 18) and to a detector 1860 through a coupler 1958. In FIG. 19, the signal goes directly to sample arm 1940 and reference arm 1942; the sampled signal is directed back through coupler 1938 and to detector 1860. The detector 1860 may use a pair of balanced photodetectors configured to cancel common mode noise. A control logic processor (control processing unit CPU) 1870 is in signal communication with tuned laser 50 and its programmable filter 1910 and with detector 1860 and obtains and processes the output from detector 1860. CPU 1870 is also in signal communication with display 1872 for command entry and for OCT results display, such as rendering of the 3D image content from various angles and sections or slices.

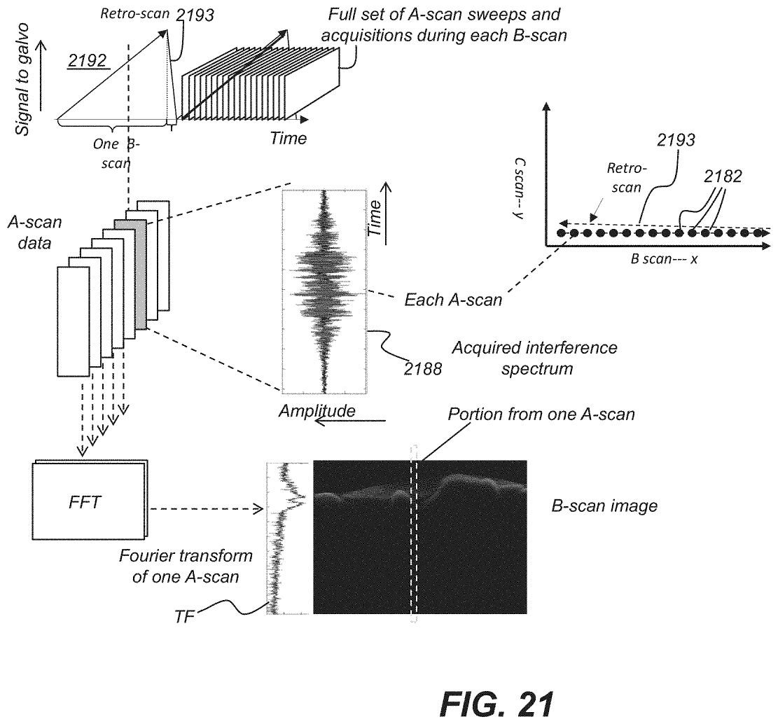

The schematic diagram of FIG. 21 shows a scan sequence that can be used for forming tomographic images of an intraoral feature using the OCT apparatus of the present disclosure. The sequence shown in FIG. 21 summarizes how a single B-scan image is generated. A raster scanner scans the selected light sequence as illumination over sample T, point by point. A periodic drive signal 2192 as shown in FIG. 21 is used to drive the raster scanner mirrors to control a lateral scan or B-scan that extends across each row of the sample, shown as discrete points 2182 extending in the horizontal direction. At each of a plurality of points 2182 along a line or row of the B-scan, an A-scan or depth scan, acquiring data in the z-axis direction, is generated using successive portions of the selected wavelength band. FIG. 20 shows drive signal 2192 for generating a straightforward ascending sequence using the raster scanner, with corresponding tuning of the laser through the wavelength band. The retro-scan signal 2193, part of drive signal 2192, simply restores the scan mirror back to its starting position for the next line; no data is obtained during retro-scan signal 2193.

It should be noted that the B-scan drive signal 2192 drives the actuable scanning mechanics, such as a galvo or a microelectro-mechanical mirror, for the raster scanner of the OCT probe 1846 (FIG. 19, 20). At each incremental scanner position, each point 2182 along the row of the B-scan, an A-scan is obtained as a type of 1D data, providing depth-resolved data along a single line that extends into the tooth. To acquire the A-scan data with spectral OCT, a tuned laser or other programmable light source sweeps through the spectral sequence. Thus, in an embodiment in which a programmable filter causes the light source to sweep through a 30 nm range of wavelengths, this sequence for generating illumination is carried out at each point 2182 along the B-scan path. As FIG. 21 shows, the set of A-scan acquisitions executes at each point 2182, that is, at each position of the scanning mirror. By way of example, there can be 2048 measurements for generating the A-scan at each position 2182.

FIG. 21 schematically shows the information acquired during each A-scan. An interference signal 2188, shown with DC signal content removed, is acquired over the time interval for each point 2182, wherein the signal is a function of the time interval required for the sweep (which has a one-to-one correspondence to the wavelength of the swept source), with the signal that is acquired indicative of the spectral interference fringes generated by combining the light from reference and feedback (or sample) arms of the interferometer (FIGS. 19, 20). The Fourier transform generates a transform TF for each A-scan. One transform signal corresponding to an A-scan is shown by way of example in FIG. 21.

From the above description, it can be appreciated that a significant amount of data is acquired over a single B-scan sequence. In order to process this data efficiently, a Fast-Fourier Transform (FFT) is used, transforming the spectral-based signal data to corresponding spatial-based data from which image content can more readily be generated.

In Fourier domain OCT, the A scan corresponds to one line of spectrum acquisition which generates a line of depth (z-axis) resolved OCT signal. The B scan data generates a 2D OCT image as a row R along the corresponding scanned line. Raster scanning is used to obtain multiple B-scan data by incrementing the raster scanner acquisition in the C-scan direction.

For ultrasound and for photo-acoustic imaging apparatus 1800, the probe 1846 transducer for signal feedback must be acoustically coupled to sample T, such as using a coupling medium. The acoustic signal that is acquired typically goes through various gain control and beam-forming components, then through signal processing for generating display data.

### Image Processing

Embodiments of the present disclosure use depth-resolved imaging techniques to help counteract the effects of fluid in intraoral imaging, allowing 3D surface reconstruction without introducing distortion due to fluid content within the intraoral cavity. In order to more effectively account for and compensate for fluid within the mouth, there remain some problems to be addressed when using the 3D imaging methods described herein.

Among problems with the imaging modalities described for 3D surface imaging is the shift of image content due to the light or sound propagation in fluid. With either OCT or ultrasound methods, the retro-reflected signals from the imaged features provide information resolvable to different depth layers, depending on the relative time of flight of light or sound. Thus the round trip propagation path length of light or sound within the fluid can cause some amount of distortion due to differences between propagation speeds of light or sound in fluid and in air.

OCT can introduce a position shift due to the refractive index difference between the surrounding fluid medium and air. The shift is 2Δnd, wherein Δn is the difference in refractive index between fluid and air, distance d is the thickness of fluid. The factor 2 is introduced due to the round trip propagation of light through distance d.

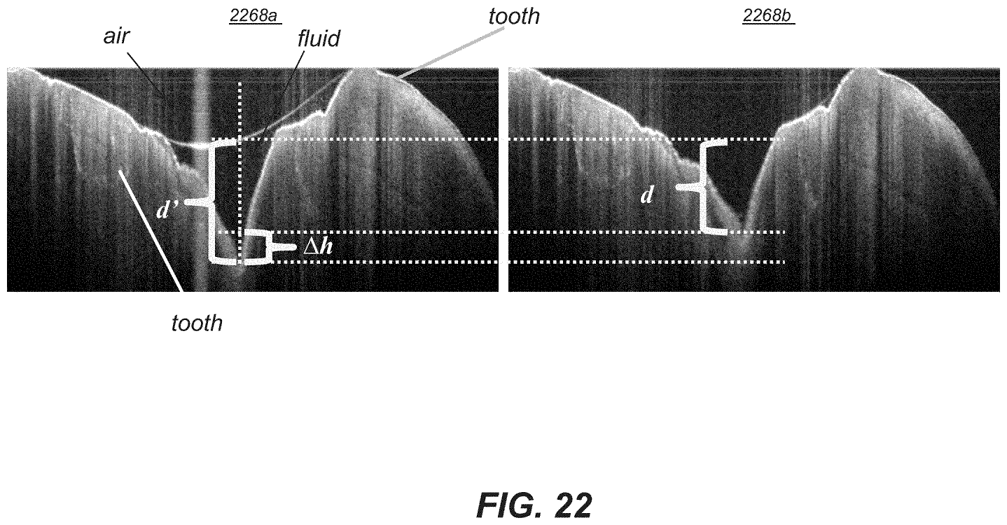

The example of FIG. 22 shows an OCT B-scan for two teeth, a first OCT scan 2268a with fluid, shown side-by-side with the corresponding scan 2268b without fluid content. As is shown in the example of FIG. 22, for the apparent height difference Δh=Δn2d in the scan 2268a, distance d′ is measured from surface point of the fluid to tooth surface point. The actual position of the tooth beneath the fluid, however, is d′/(1+Δn), for example (d′/1.34 for water).

Similarly, ultrasound has a shift effect caused by a change in the speed of sound in the fluid. The calculated shift is Δc×2d, wherein Δc is the speed difference of sound between air and fluid.

Photoacoustics imaging relies on pulsed light energy to stimulate thermal extension of probed tissue in the sample. The excitation points used are the locations of the acoustic sources. Photoacoustics devices capture these acoustic signals and reconstruct the 3D depth resolved signal depending on the receiving time of sound signals. If the captured signal is from the same path of light, then the depth shift is Δc×d, where Δc is the speed difference of sound between air and fluid. Value d is the thickness of fluid.

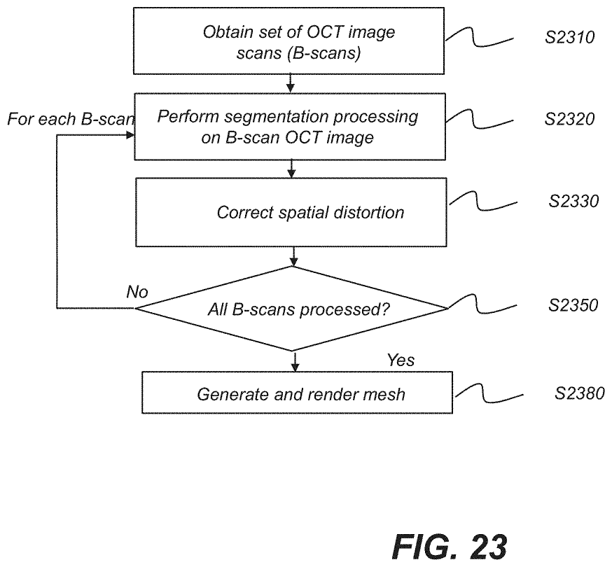

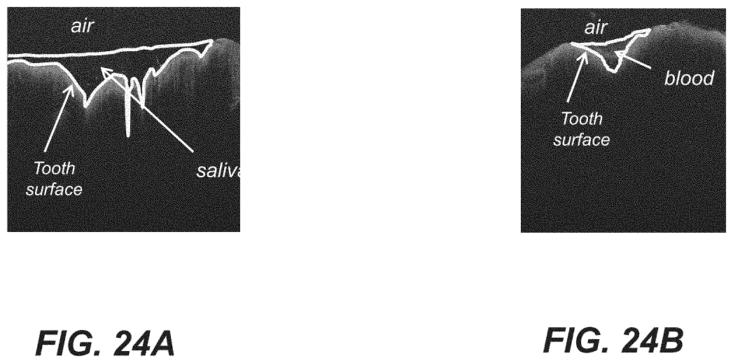

The logic flow diagram of FIG. 23 shows a processing sequence for fluid compensation using OCT imaging. In an acquisition step S2310, a set of OCT image scans is obtained. Each element in the set is a B-scan, or side-view scan, such as the scans shown in FIG. 22, for example. The block of steps that follows then operates on each of the acquired B-scans. A segmentation step S2320 identifies fluid and tooth surfaces from the B-scan image, by detecting multiple interfaces as shown in the schematic diagram of FIG. 1. Segmentation step S2320 defines the tooth surface and the area of the B-scan image that contains intraoral fluid such as water, saliva, or blood, as shown in the example of FIGS. 24A and 24B. Then, in order to obtain more accurate characterization of the 3D surfaces, a correction step S2330 corrects for spatial distortion of the tooth surface underneath the fluid due to refractive index differences between air and the intraoral fluid. Step S2330 adjusts the measured depth of segmented regions in the manner discussed above, based on the thickness of the region and refractive index of the fluid within the region. For example, the refractive index of water for the OCT illumination is approximately 1.34; for blood in a 50% concentration, the refractive index is slightly higher, at about 1.36.

The thickness of the region is determined through a calibrated relationship between the coordinate system inside the OCT probe and the physical coordinates of the teeth, dependent on the optical arrangement and scanner motion inside the probe. Geometric calibration data are obtained separately by using a calibration target of a given geometry. Scanning of the target and obtaining the scanned data establishes a basis for adjusting the registration of scanned data to 3D space and compensating for errors in scanning accuracy. The calibration target can be a 2D target, imaged at one or more positions, or a 3D target.

The processing carried out in steps S2320 and S2330 of FIG. 23 is executed for each B-scan obtained by the OCT imaging apparatus. A decision step S2350 then determines whether or not all B-scans in the set have been processed. Once processing is complete for the B-scans, the combined B-scans form a surface point cloud for the teeth. A mesh generation and rendering step S2380 then generates and renders a 3D mesh from the surface point cloud. The rendered OCT surface data can be displayed, stored, or transmitted.

Various image segmentation algorithms can be used for the processing described with relation to FIG. 23, including simple direct threshold, active contour level set, watershed, supervised and unsupervised image segmentation, neural network based image segmentation, spectral embedding, k-means, and max-flow/min-cut graph based image segmentation, for example. Segmentation algorithms are well known to those skilled in image processing and can be applied to the entire 3D volume, reconstructed from the OCT data, or applied separately to each 2D frame or B-scan of the tomographic data prior to 3D volume reconstruction, as described above.

Processing for photoacoustics and ultrasound imaging is similar to that shown in FIG. 23, with appropriate changes for the signal energy that is detected.

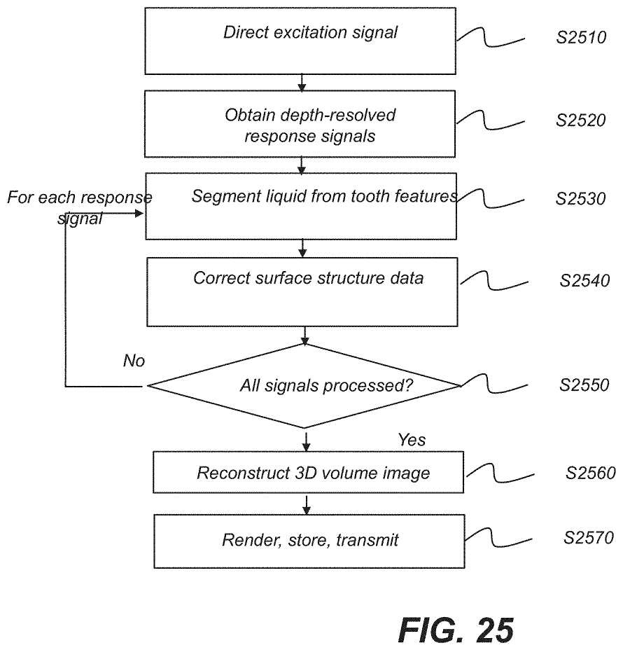

The logic flow diagram of FIG. 25 shows a sequence that can be used for imaging a tooth surface according to an embodiment of the present disclosure. In a signal excitation step S2510, an excitation signal is directed toward the subject tooth from a scan head, such as an OCT probe or a scan head that directs light for a photoacoustic imaging apparatus or sound for an ultrasound apparatus. An acquisition step S2520 acquires the depth-resolved response signal that results. The depth-resolved response signal can be light or sound energy, for example, that encodes information about the structure of the tooth surface. A segmentation step S2530 then segments liquid from tooth and gum features from the depth-resolved response signal. Surface structure information from the depth-resolved response signal can then be corrected using the segmentation data in an adjustment step S2540. A looping step S2550 determines whether or not additional depth-resolved response signals must be processed. A reconstruction step S2560 then reconstructs a 3D image of the tooth according to the depth-resolved response signal and the adjusted tooth surface structure information. A rendering step S2570 then renders the volume image content for display, transmission, or storage.

Consistent with one embodiment, the present disclosure utilizes a computer program with stored instructions that control system functions for image acquisition and image data processing for image data that is stored and accessed from an electronic memory. As can be appreciated by those skilled in the image processing arts, a computer program of an embodiment of the present disclosure can be utilized by a suitable, general-purpose computer system, such as a personal computer or workstation that acts as an image processor, when provided with a suitable software program so that the processor operates to acquire, process, and display data as described herein. Many other types of computer systems architectures can be used to execute the computer program of the present disclosure, including an arrangement of networked processors, for example. The computer program for performing the method of the present disclosure may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present disclosure may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present disclosure may make use of various image manipulation algorithms and processes that are well known. It will be further understood that the computer program product embodiment of the present disclosure may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present disclosure, are not specifically shown or described herein and may be selected from such algorithms, systems, hardware, components and elements known in the art.

Exemplary embodiments according to the application can include various features described herein, individually or in combination.

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. Other embodiments of the invention will be apparent to those skilled in the art from consideration of the specification and practice of the invention disclosed herein. It is intended that the specification and examples be considered as exemplary only, with a true scope and spirit of the invention being indicated by the following claims, and all changes that come within the meaning and range of equivalents thereof are intended to be embraced therein.

1. A method for acquiring and updating a 3-D surface of a dentition,
the method executed at least in part by a computer and comprising:
a) acquiring a collection of 3-D image content of the dentition from different points of view using a 3-D scanning device;
b) gradually forming the 3-D surface of the dentition using a matching algorithm that aggregates 3-D images from the 3-D image content based on a determination of overlap of each 3-D image relative to the 3-D surface of the dentition;
wherein for each newly acquired 3-D image,
i) when the newly acquired 3-D image partly overlaps with the 3-D surface of the dentition, augmenting the 3-D surface of the dentition with a portion of the newly acquired 3-D image that does not overlap with the 3-D surface of the dentition, and
ii) when the newly acquired 3-D image completely overlaps with the 3-D surface of the dentition, updating the 3-D surface of the dentition in real time by replacing the corresponding portion of the 3-D surface of the dentition with the contents of newly acquired 3-D image, where the corresponding portion of the 3-D surface of the dentition no longer contributes to the updated 3-D surface of the dentition.

2. The method of claim 1, comprising:
determining the position of the 3-D scanning device relative to the 3-D surface of the dentition in real time by comparing the size and the shape of the overlap to the cross-section of the field-of-view of the 3-D scanning device,
where the size and the shape of the overlap of the newly acquired 3-D image is used to determine the distance and the angles from which the 3-D image was acquired relative to the 3-D surface of the dentition.

3. The method of claim 1, further comprising:
updating the 3-D image content according to a plurality of structured light images of the dentition; and
displaying the updated 3-D image content.

4. The method of claim 1, further comprising
updating the 3-D surface of the dentition according to a plurality of images of a 3-D site of interest of the dentition from each of a plurality of cameras of a 3-D intra-oral scanner, and
displaying the updated 3-D surface of the dentition.

5. The method of claim 1, further comprising:
physically modifying a 3-D treatment region of the dentition using a dental instrument; and
updating the 3-D treatment region in the 3-D surface of the dentition according to a plurality of structured light images of the 3-D treatment region obtained from the 3-D scanning device mounted to the dental instrument.

6. The method of claim 2, further comprising:
positioning a dental surgical instrument mounted to the 3-D scanning device at a 3-D site of interest within a 3-D treatment region of the dentition;
detecting and monitoring a field of view from a head-mounted device worn by a practitioner, and
displaying the 3-D site of interest within the 3-D treatment region in the detected field of view on a display of the head-mounted worn by a practitioner.

7. The method of claim 6, comprising:
displaying the updated 3-D surface of the dentition, by:
physically modifying the 3-D site of interest using the dental surgical instrument;
updating the 3-D site of interest within the treatment region of the 3-D surface of the dentition according to a plurality of structured light images of the 3-D site of interest obtained from the 3-D scanning device mounted to the dental surgical instrument; and
displaying the updated 3-D site of interest of the 3-D surface of the dentition superimposed on the field of view of the head-mounted display worn by the practitioner; and
displaying features of a surgical treatment plan within the practitioner's field of view.

8. The method of claim 7, wherein displaying the updated 3-D site of interest of the 3-D surface of the dentition to the practitioner comprises forming a virtual image oriented to the monitored field of view of the head-mounted display worn by the practitioner.

9. The method of claim 1, comprising:
a) acquiring 3-D image content of the dentition from a volume radiographic imaging apparatus that obtains a plurality of radiographic images at differing angles;
b) combining the acquired radiographic 3-D image content with the 3-D surface of the dentition to form a 3-D mesh model of the subject dentition;
c) updating the 3-D mesh model according to changes made to a surface contour at a site location on the dentition using a dental instrument; and
d) displaying the updated 3-D mesh model.

10. The method of claim 9, comprising repeating the updating and displaying during a treatment of the site location on the dentition according to a dental treatment plan.

11. The method of claim 10, comprising registering the updated 3-D mesh model to a field of view of a viewer to according to one or more additional cameras worn by the viewer.

12. The method of claim 9, comprising:
forming a second 3-D surface of the dentition using the radiographic 3-D image content; and
combining the radiographic 3-D image content with the 3-D surface of the dentition to display a 3-D volume of the dentition and the 3-D surface of the dentition.

13. The method of claim 1, where acquiring a collection of 3-D image content comprises:
directing an excitation signal toward the tooth from a scan head;
obtaining a depth-resolved response signal emanating from the tooth, wherein the response signal encodes tooth surface structure information;
segmenting liquid and tooth surface from the depth-resolved response signal;
adjusting the tooth surface structure information based on the segmented liquid; and
reconstructing a 3D image of the tooth according to the depth-resolved response signal and the adjusted tooth surface structure information.

14. The method of claim 1 wherein the excitation signal is a broadband light signal, a pulsed or modulated laser source, or an acoustic signal.

15. A method for updating display of a dentition to a practitioner, the method executed at least in part by a computer and comprising:
obtaining 3-D surface contour image content that comprises a dentition treatment region;
obtaining radiographic volume image content that comprises the dentition treatment region;
combining the 3-D surface contour image content and the radiographic volume image content into a single 3-D virtual model that comprises the dentition treatment region;
obtaining instructions that define a surgical treatment plan related to the treatment region;
repeating the steps of:
a1) acquiring new 3-D contour images of the dentition treatment region that comprise physical dental objects in the dentition treatment region from different points of view using a 3-D scanning device, and
a2) updating the 3-D surface of the dentition treatment region in real time by replacing the corresponding portion of the 3-D surface of the dentition treatment region with the contents of the newly acquired 3-D contour images, where the corresponding portion of the 3-D surface of the dentition no longer contributes to the updated 3-D surface of the dentition; and
repeating the steps of:
(b1) sensing the position of a surgical instrument mounted to the 3-D scanning device at a surgical site within the dentition treatment region, relative to the single 3-D virtual model;
b2) updating the single 3-D virtual model according to the surgical treatment plan;
b3) determining a field of view of the practitioner and detecting a tooth surface in the dentition treatment region in the practitioner's field of view and displaying at least a portion of the updated single 3-D virtual model onto the field of view and oriented to the field of view and registered to the actual tooth surface as seen from the practitioners' field of view.

16. The method of claim 15, where the updated single 3-D virtual model oriented to the field of view and registered to the actual tooth surface as seen from the practitioners' field of view is displayed in the practitioners' field of view at the position, size and orientation of the actual tooth surface.

17. The method of claim 15, comprising:
determining the position of the 3-D scanning device relative to the 3-D surface of the dentition treatment region in real time by comparing the size and the shape of the replaced corresponding portion of the 3-D surface of the dentition treatment region to the cross-section of the field-of-view of the 3-D scanning device,
where the size and the shape of the overlap of the newly acquired 3-D image is used to determine the distance and the angles from which the 3-D image was acquired relative to the 3-D surface of the dentition treatment region.

18. The method of claim 15 wherein displaying the registered updated single 3-D virtual model comprises:
displaying features of the surgical treatment plan within the practitioner's field of view; and
refreshing the registered updated single 3-D virtual model according to the updated 3-D surface of the dentition.

19. The method of claim 15 further comprising refreshing the registered updated single 3-D virtual model comprises displaying a status indicator for the practitioner, and where the updated single 3-D virtual model further includes image content that is representative of the position of a surgical instrument.

20. The method of claim 15, where obtaining 3-D surface contour image content that comprises a dentition treatment region comprises acquiring surface contour image content of the dentition treatment region according to a plurality of structured light images, and
where obtaining radiographic volume image content that comprises the dentition treatment region comprises determining volumetric 3-D image content of the subject dentition and surface contour 3-D image content of the subject dentition from a volume radiographic imaging apparatus that obtains a plurality of radiographic images at differing angles.

21. The method of claim 15, where displaying the registered updated single 3-D virtual model comprises directing the image content to a planar waveguide that is worn by the practitioner, and wherein detecting the treatment region in the practitioner's field of view comprises coupling cameras to a head-mounted device, registering at least a portion of the updated single 3-D virtual model onto the field of view using a head-mounted display, and superimposing at least a portion of the surgical treatment plan at a periphery of the field of view.

22. A method for updating display of a dentition to a practitioner, the method executed at least in part by a computer and comprising:
obtaining 3-D surface contour image content that comprises a dentition treatment region;
obtaining radiographic volume image content that comprises the dentition treatment region;
combining the 3-D surface contour image content and the radiographic volume image content into a single 3-D virtual model that comprises the dentition treatment region;
detecting the dentition treatment region in the practitioner's field of view and displaying at least a portion of the single 3-D virtual model superimposed onto the field of view and oriented to the field of view, where the superimposed portion of the single 3-D virtual model in the practitioners' field of view is registered to the actual object as seen from the practitioners' field of view;
obtaining instructions that define a surgical treatment plan related to the dentition treatment region;
repeating the steps of:
a1) updating the 3-D surface of the dentition treatment region in real time by replacing the corresponding portion of the 3-D surface of the dentition treatment region with contents of newly acquired 3-D images of the dentition treatment region that comprise physical dental objects in the dentition treatment region from different points of view using a 3-D intra-oral scanning device, where the corresponding portion of the 3-D surface of the dentition no longer contributes to the updated 3-D surface of the dentition;
(a2) sensing the position of a surgical instrument mounted to the 3-D intra-oral scanning device at a surgical site within the dentition treatment region, relative to the single 3-D virtual model;
(a3) updating the superimposed single 3-D virtual model onto the field of view registered to the actual object as seen from the practitioners' field of view according to the surgical treatment plan and the updated 3-D surface of the dentition treatment region; and
(a4) providing deviation information to the practitioner superimposed onto the field of view and oriented to the field of view when the sensed position of a surgical instrument is contrary to the surgical treatment plan.

23. The method of claim 22, where the deviation information is an orientation of the surgical instrument and correction information in accordance with the surgical treatment plan displayed in the practitioners' field of view registered to the actual object as seen from the practitioners' field of view.

24. The method of claim 22, where one or more cameras obtain image content of the dentition treatment region from the practitioner's field of view, where a surgical instrument camera is coupled to the surgical instrument, and where the surgical instrument is a dental drill.

25. A method, comprising:
a) obtaining a 3-D surface of dentition, the dentition comprising at least physical teeth or gums of a patient; and
b) acquiring a collection of 3-D images of the dentition from different points of view using a 3-D scanning device,
wherein for each newly acquired 3-D image,
when the newly acquired 3-D image at least partly overlaps with the 3-D surface of the dentition,
determining the position of the 3-D scanning device relative to the physical dentition represented by the 3-D surface of the dentition in real time by comparing the size and the shape of the overlap to the cross-section of the field-of-view of the 3-D scanning device, and
where the size and the shape of the overlap of the newly acquired 3-D image is used to determine the distance and the angles from which the 3-D image was acquired relative to the 3-D surface of the dentition.

26. The method of claim 25, comprising:
augmenting the 3-D surface of the dentition using a matching algorithm that aggregates 3-D images from the 3-D image content based on a determination of overlap of each 3-D image relative to the 3-D surface of the dentition;
wherein for said each newly acquired 3-D image,
i) when the newly acquired 3-D image partly overlaps with the 3-D surface of the dentition, augmenting the 3-D surface of the dentition with a portion of the newly acquired 3-D image that does not overlap with the 3-D surface of the dentition, and
ii) when the newly acquired 3-D image completely overlaps with the 3-D surface of the dentition, updating the 3-D surface of the dentition in real time by replacing the corresponding portion of the 3-D surface of the dentition with the contents of newly acquired 3-D image, where the corresponding portion of the 3-D surface of the dentition no longer contributes to the updated 3-D surface of the dentition.
